(19) United States
(12) Patent Application Publication
(10) Pub. No.: US 2025/0188141 A1
(43) Pub. Date: Jun. 12, 2025

(54) DECOY PEPTIDES FOR TREATING DISEASES OR CONDITIONS MODULATED BY INTERLEUKIN-33

(71) Applicants: University of Maryland, Baltimore, Baltimore, MD (US); The United States Government as represented by the Department of Veterans Affairs, Washington, DC (US)

(72) Inventors: Sergei P. Atamas, Ellicott City, MD (US); Irina G. Luzina, Ellicott City, MD (US)

(21) Appl. No.: 18/849,417

(22) PCT Filed: Mar. 22, 2023
(86) PCT No.: PCT/US2023/064809

§ 371 Date: Sep. 20, 2024

Related U.S. Application Data
(60) Provisional application No. 63/322,484, filed on Mar. 22, 2022.

(51) Int. Cl. C07K 14/54; A61K 38/00; C12N 15/86

(52) U.S. Cl. CPC C07K 14/54; C12N 15/86; A61K 38/00; C07K 2319/21

(57) ABSTRACT

The present invention provides an IL-33 decoy peptide comprising an amino acid sequence of a fragment of FLIL33 or a fragment of a FLIL33 variant, wherein the decoy peptide disrupts an association of FLIL33 with an FLIL33 binding partner.

### CROSS-REFERENCE TO RELATED APPLICATIONS

This application claims the benefit of U.S. Provisional Appl. No. 63/322,484, filed on Mar. 22, 2022, the contents of which are hereby incorporated by reference in their entirety.

### STATEMENT OF FEDERALLY SPONSORED RESEARCH AND DEVELOPMENT

This invention was made with government support under Grant Number AR077562 awarded by The National Institutes of Health. The government has certain rights in the invention.

### FIELD OF THE INVENTION

The field of the invention relates to medicine and pharmaceuticals, particularly methods and compositions for treating inflammation and fibrosis.

### BACKGROUND

Fibrosis is an essential process that is a critical part of wound healing. Excessive fibrosis is common in many rare and common disease conditions and is important in disease pathogenesis. Diseases characterized by excessive fibrosis include but are not restricted to systemic sclerosis, scleroderma, hypertrophic cardiomyopathy, dilated cardiomyopathy (DCM), atrial fibrillation, ventricular fibrillation, myocarditis, liver cirrhosis, kidney diseases, diseases of the eye, asthma, cystic fibrosis, arthritis and idiopathic pulmonary fibrosis. Despite the large impact on human health, therapeutic and diagnostic approaches to fibrosis are still an unmet medical need.

Inflammation and fibrosis are distinct yet frequently co-occurring processes contributing to a remarkably broad variety of diseases of every organ and tissue, including skin, heart, lung, liver, gut, kidney, peritoneum, adipose tissue, and skeletal muscle (Nystrom A, Matrix Biol, (2018), 68-69:547-60; Artlett, Do they have a role in scleroderma fibrosis? Immunol Lett, (2018), 195:30-7: Chizzolini et al., Autoimmun Rev 2011, 10:276-81: Prabhu et al., Circ Res, (2016), 119:91-112; Desai et al., Front Med (Lausanne), (2018), 5:43; Borthwick et al., Semin Immunopathol, (2016), 38:517-34; Luzina et al., Cytokine, (2015), 74:88-100; Koyama et al., J Clin Invest, (2017), 127:55-64; Bettenworth et al., Dig Dis, (2017), 35:25-31; Curciarello et al., Front Med (Lausanne), (2017), 4:126; Meng et al., Nat Rev Nephrol, (2014), 10:493-503: Witowski et al., Biomed Res Int, (2015), 2015:134708; Crewe et al., J Clin Invest. (2017), 127:74-82; Alameddine et al., J Neuromuscul Dis. (2016). 3:455-73). The association between inflammation and fibrosis is commonly acknowledged across medical disciplines, and a causal link has been suggested, although anti-inflammatory therapies are not curative in many fibrotic diseases. It appears that the link between inflammation and fibrosis extends beyond association or causation to the shared molecular mechanisms that mediate both of these fundamental processes. Specifically, the same cytokines and cell-surface molecules that mediate inflammation also contribute to fibrosis, suggesting that in-depth understanding of these shared mediators forms the basis for novel therapeutic approaches to attenuate both conditions (Artlett. Do they have a role in scleroderma fibrosis? Immunol Lett. (2018), 195:30-7; Borthwick et al., Semin Immunopathol, (2016), 38:517-34; Luzina et al., Cytokine, (2015). 74:88-100; Curciarello et al., Front Med (Lausanne), (2017), 4:126; Sziksz et al., Mediators Inflamm, (2015), 2015:764641; Borthwick et al., Biochim Biophys Acta, (2013), 1832:1049-60). IL-33 has recently emerged as a key mediator of both inflammation and fibrosis, as well as an attractive therapeutic target, yet there are no approved therapies that manipulate IL-33. The complexities of its biology and pathology need to be better understood for rational development of novel therapies (Cayrol et al., Immunol Rev, (2018), 281:154-68; Zhao et al., J Pathol, (2018): Vannella et al., Sci Transl Med, (2016), 8: 337ra65: Martin et al., Nat Immunol, (2016), 17:122-31: Liew et al., Nat Rev Immunol, (2016), 16:676-89; De la Fuente et al., Cytokine Growth Factor Rev, (2015), 26:615-23; Molofsky et al., Immunity, (2015), 42:1005-19; Li D et al., J Allergy Clin Immunol, (2014), 134:1422-32 ell: McHedlidze et al., Immunity. (2013), 39:357-71; Rankin A L et al., J Immunol, (2010), 184:1526-35: Tajima et al., Chest, (2003), 124:1206-14; Licona-Limon et al., Nat Immunol, (2013). 14:536-42).

Its precursor, full-length IL-33 (FLIL33), behaves distinctly from proteolytically activated, mature IL-33 (MIL33). FLIL33 is basally and inducibly expressed, resides mostly in the nucleus but also in the cytoplasm of a wide variety of abundant cell types, and modulates inflammatory responses, wound healing, chromatin stability, and transcriptional regulation in a non-Th2 and receptor-independent manner (Cayrol et al., Immunol Rev, (2018), 281:154-68; Martin et al., Nat Immunol. (2016), 17:122-31; Liew et al., Nat Rev Immunol. (2016). 16:676-89; De la Fuente et al., Cytokine Growth Factor Rev, (2015), 26:615-23: Molofsky et al., Immunity, (2015), 42:1005-19; Byers et al., J Clin Invest, (2013), 123:3967-82: Pichery et al., J Immunol, (2012), 188:3488-95: Ali et al., J Immunol, (2011), 187: 1609-16; Clerman et al., J Biol Chem, (2017), 292: 21653-61: Kopach et al., J Biol Chem, (2014), 289:11829-43; Luzina et al., Am J Respir Cell Mol Biol, 2013, 49:999-1008; Luzina et al., J Immunol, (2012), 189:403-10; Carriere et al., Proc Natl Acad Sci USA, (2007), 104:282-7). MIL33 is a powerful pro-Th2 cytokine acting through its specific cell-surface receptor ILIRLI (a.k.a. T1/ST2 or ST2) (Cayrol et al., Immunol Rev. (2018), 281: 154-68; Martin et al., Nat Immunol, (2016), 17: 122-31: Liew et al., Nat Rev Immunol, (2016). 16:676-89; De la Fuente et al., Cytokine Growth Factor Rev, (2015), 26:615-23: Molofsky et al., Immunity, (2015), 42:1005-19; Licona-Limon et al., Nat Immunol, (2013), 14:536-42). The MIL33-ST2 axis is better characterized.

Unlike the majority of cytokines that are released by source cells when needed, and similar only to IL-la and HMGB1, IL-33 is constitutively present and also induced as the intracellular (mostly intranuclear but possibly partially cytoplasmic) precursor (FLIL33) (Kakkar et al., J Biol Chem, (2012), 287:6941-8). It is thought that IL-33 is released from necrotic cells as FLIL33, which becomes proteolytically activated extracellularly into MIL33, binds to its cell-surface receptor ST2 on target cells, activates intracellular signaling, and elicits functional effects, acting as an alarmin and a powerful inducer of the Th2 phenotype (Cayrol et al., Immunol Rev, (2018), 281:154-68; Martin et al., Nat Immunol, (2016). 17:122-31: Molofsky et al., Immunity, (2015), 42:1005-19; Licona-Limon et al., Nat Immunol. (2013), 14:536-42; Cayrol et al., Nat Immunol, (2018), 19:375-85; Lefrancais et al., Proc Natl Acad Sci USA, (2014), 111: 15502-7; Lefrancais et al., Proc Natl Acad Sci USA, (2012), 109:1673-8). A sizeable body of evidence indicates that IL-33 may also be actively secreted from live cells, including in its active, MIL33 form, although the mechanisms of such secretion are completely unknown (Kakkar et al., J Biol Chem, (2012), 287:6941-8: Hristova et al., J Allergy Clin Immunol, (2016), 137:1545-56 ell; Lee et al., Biochem Biophys Res Commun, (2015), 464:20-6; et al., J Interferon Cytokine Res. (2014), 34:141-7; Lefrancais et al., Eur Cytokine Netw. (2012), 23:120-7; Balato et al., Exp Dermatol, (2012), 21:892-4). The intracellular precursor FLIL33 is also independently active and contributes to injury responses and to the pathophysiology of diseases (Cayrol et al., Immunol Rev, (2018), 281:154-68; Martin et al., Nat Immunol, (2016), 17:122-31; Molofsky et al., Immunity, (2015), 42:1005-19; Byers et al., J Clin Invest, (2013), 123:3967-82; Pichery et al., J Immunol, (2012), 188:3488-95: Ali et al., J Immunol, (2011), 187:1609-16; Clerman et al., J Biol Chem, (2017), 292:21653-61; Kopach et al., J Biol Chem, (2014), 289:11829-43; Luzina et al., Am J Respir Cell Mol Biol, 2013, 49:999-1008: Luzina et al., J Immunol, (2012), 189:403-10: Carriere et al., Proc Natl Acad Sci USA. (2007), 104:282-7). Overall, the complex IL-33 biology involves the interplay between its transcriptional regulation, the protein pool stability inside cells, the regulation of subcellular distribution and intracellular signaling from FLIL33, and the active secretion or necrotic release and proteolytic activation of the MIL33 cytokine acting on ST2-expressing target cells. The regulation of these processes is poorly understood.

An early study roughly identified the region amino acids 60-80 as responsible for the nuclear localization of FLIL33, although the authors did not precisely pinpoint the specific amino acids responsible for nuclear trafficking, the exact boundaries of the region, or, most importantly, the relationship of this region to IL-33 secretion or activation into MIL33 (Carriere et al., Proc Natl Acad Sci USA, (2007), 104: 282-7). They later identified the region aa 95-109 as the “sensor domain” responsible for the proteolytic activation of FLIL33 into MIL33 variants of various lengths, but did not explore the potential of the more N-terminal region to contribute to IL-33 maturation (Cayrol et al., Nat Immunol, (2018), 19:375-85: Lefrancais et al., Proc Natl Acad Sci USA, (2014), 111:15502-7; Lefrancais et al., Proc Natl Acad Sci USA, (2012), 109:1673-8).

What is needed are new compositions and methods for treating diseases or conditions, such as inflammation and fibrosis, that are modulated by IL-33. This background information is provided for informational purposes only. No admission is necessarily intended, nor should it be construed, that any of the preceding information constitutes prior art against the present invention.

### SUMMARY

It is to be understood that both the foregoing general description of the embodiments and the following detailed description are exemplary, and thus do not restrict the scope of the embodiments.

The invention relates to methods of depleting FLIL33 for therapeutic purposes. FLIL33 is both an independently active intracellular factor and the source of the MIL33 cytokine. In some embodiments, the methods attenuate inflammation and/or fibrosis. By targeting FLIL33 and thus diminishing the entire IL-33 pool, including FLIL33 and MIL33, this method offers a key advantage over MIL33-targeting and ST2-targeting approaches, which do not eliminate the continuous supply of MIL33 from the FLIL33 precursor form.

According to non-limiting example embodiments, in one aspect, the invention provides an IL-33 decoy peptide comprising an amino acid sequence of a fragment of FLIL33 or a fragment of a FLIL33 variant, wherein the decoy peptide disrupts an association of FLIL33 with an FLIL33 binding partner. In some embodiments, the decoy peptide disrupts an association of FLIL33 with IPO5.

In some embodiments, the decoy peptide further comprises an amino acid sequence that enables the peptide to enter a cell.

In some embodiments, the decoy peptide comprises an amino acid sequence of a fragment of the N-terminal domain of FLIL33 or a fragment of the N-terminal domain of a FLIL33 variant.

In some embodiments, the peptide sequence comprises an amino acid sequence selected from the group consisting of any one of SEQ ID NOS: 5-14.

In some embodiments, the fragment of FLIL33 is 10-50 amino acids. In some embodiments, the fragment of FLIL33 is 15-30 amino acids.

In some embodiments, the amino acid sequence that enables the peptide to enter a cell is selected from any one of SEQ ID NOS: 15-61.

In some embodiments, the IL-33 decoy peptide comprises one or more epitope tags and/or purification tags. In some embodiments, the epitope tag is selected from the group consisting of a Myc tag, a FLAG tag, a hemagglutinin (HA) tag and combinations thereof. In some embodiments, the purification tag is selected from the group consisting of a histidine tag (6x), a glutathione S-transferase tag and a combination thereof. In some embodiments, the IL-33 decoy peptide further comprises an enzymatic cleavage site.

In another aspect, the invention provides a nucleic acid molecule encoding an IL-33 decoy peptide.

In another aspect, the invention provides a viral vector encoding an IL-33 decoy peptide.

In another aspect, the invention provides a pharmaceutical composition comprising an IL-33 decoy peptide, a nucleic acid encoding the same, or a viral vector encoding the same, and one or more pharmaceutically acceptable excipients.

In another aspect, the invention provides a method of a method of treating and/or preventing a disease or condition that is modulated by IL-33 in a subject in need thereof, comprising administering to the subject an effective amount of a pharmaceutical composition comprising an IL-33 decoy peptide, a nucleic acid encoding the same, or a viral vector encoding the same. In some embodiments, the disease or condition is an infection. In some embodiments, the disease or condition is a viral infection. In some embodiments, the disease or condition is a metabolic disorder. In some embodiments, the disease or condition is characterized by inflammation. In some embodiments, the disease or condition is characterized by fibrosis.

In some embodiments, the decoy peptide disrupts an association of FLIL33 with importin 5 (IPO5).

It is to be understood that both the foregoing general description and the following detailed description are exemplary, and thus do not restrict the scope of the invention. Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Functional activities of full-length IL-33 (FLIL33) are sequentially distributed along the primary structure. The C-terminal portion of the molecule is released as mature IL-33 (MIL33) cytokine following proteolytic cleavage at any of multiple sites within the sensor, or activation, domain. The N-terminal region is responsible for intracellular localization, protein stability, and numerous intracellular functions discussed in this proposal. Targeting this region is also proposed for therapeutic effect.

FIG. 2. Design of decoy peptides in Series 2 (A), the effects of peptides 2-5 and 2-10 on pulmonary levels of IL-33 (B, D), and collagen (C, D) proteins in the acute BLM model in vivo. In panels B and D, mean ±SD values are shown. The scatterplot in panel D shows the same IL-33 and collagen level values in each mouse. Wild-type C57BL/6 mice were challenged with a single intratracheal instillation of 0.075 U of BLM or PBS vehicle, and the disease was allowed to evolve for 5 days. On days 5-13, mice received daily intraperitoneal injections of 10 nmol/g of peptides 2-5 or 2-10, or PBS vehicle. Mice were euthanized on day 14, and IL-33 levels measured by ELISA in lung homogenates, whereas total lung collagen was measured using QuickZyme assays. Note that therapy with peptide 2-5 but not 2-10 attenuated the levels of IL-33 and arrested deposition of collagen.

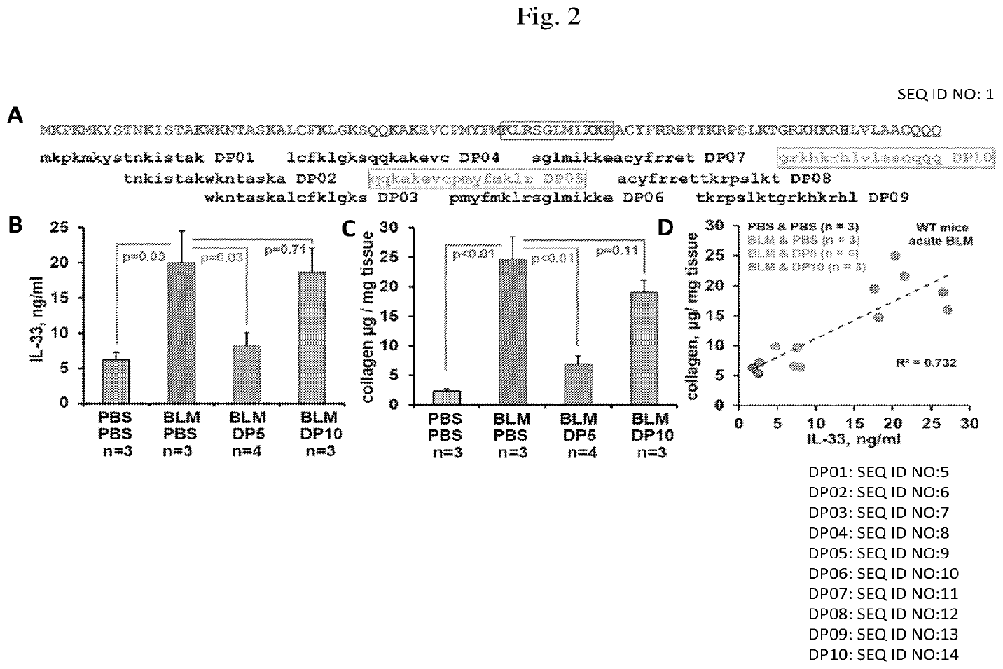

FIG. 3. Importing-5 binding segment (underlined bold capital letters) in the N-terminal portion of FLIL33 (top). Amino acid sequence numbers are indicated in the second line from the top. Each of the decoy peptides DP5 (SEQ ID NO:9) and DP7 (SEQ ID NO:11) are predicted to compete with FLIL33 for importin-5 binding, whereas control peptide DP10 (SEQ ID NO:14) is not. Amino acids 31-90 of SEQ ID NO: 3 (FLIL33) are shown.

FIG. 4. Amino acid sequences of indicated CPP-DPs. The CPP portion is shown in italic.

FIG. 5. Therapeutic effects of CPP-DPs in the acute BLM challenge model. A. Time course schematic of the acute BLM challenge model. B. Time-dependent changes in total body weight (mean percent of initial body weight ±SD. Asterisks indicate higher (p<0.05) body weights in CPP-DP-treated compared to BLM-challenged, PBS-treated control mice. C. Individual collagen levels, μg per mg of wet lung tissue (top left), and IL-33 levels, pg per mg of wet lung tissue (top right), in lung homogenates from mice treated as indicated. Asterisks indicate lower (p<0.05) levels in BLM-challenged. PBS-treated control mice. The scatterplot at the bottom shows correlation between individual lung collagen and pulmonary IL-33 levels using the same data across the experimental groups, with each circle representing a separate mouse. Data in panels B and C were pooled data from separate 3 experiments.

FIG. 6. Therapeutic effects of CPP-DPs in the chronic BLM challenge model. A. Time course schematic of the chronic BLM challenge model. B. Time-dependent changes in total body weight (mean percent of initial body weight ±SD. Asterisks indicate higher (p<0.05) body weights in CPP-DP-treated compared to PBS control-treated mice. C. Individual collagen levels, μg per mg of wet lung tissue (left), and IL-33 levels, pg per mg of wet lung tissue (right), in lung homogenates from mice treated as indicated. Asterisks indicate lower (p<0.05) levels in BLM-challenged, PBS-treated control mice.

### DETAILED DESCRIPTION

The present invention is based, at least in part, on the discovery of previously unknown structure-function relationships in the N-terminal portion of the FLIL33 that can be therapeutically targeted.

The present invention also describes cell-permeable peptides, which can be used to deplete the FLIL33 protein pool to both attenuate the functional effects of intracellular FLIL33 and exhaust the source of the proteolytically mature IL-33 cytokine. The peptides act as decoys by mimicking sequences found in IL-33 that control key aspects of FLIL33 biology and pathology. These include nuclear localization, extracellular secretion, and proteolytic activation of IL-33, as well as binding of various intracellular molecular partners. Such binding affects the functioning of both FLIL33 and its intracellular ligands. The broad and defining contribution of IL-33 to numerous diseases renders it a target therapeutically for the decoy peptides herein. In diseases, the ever-present intracellular pool of FLIL33 protein continuously supplies new MIL33 as well as acts independently as an intracellular mediator. The decoy peptides herein can be used to control the levels of both FLIL33 and MIL33.

The present invention is advantageous over other potential therapies such as antibodies which target IL-33 or its receptor ST2. Neutralization of the continuous release of new MIL33 would require substantial amounts of antibody or frequent injections of soluble ST2 receptor, thus diminishing the feasibility of such an approach. Furthermore, intracellular FLIL33, which contributes pathophysiologically on its own, is completely inaccessible to antibodies or soluble ST2. The decoy peptides herein are cell permeable or can be delivered therapeutically inside cells and can control the intracellular protein pool of FLIL33, thus simultaneously depleting the source of MIL33 and attenuating the independent effects of FLIL33.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

### Decoy Peptides

In some embodiments, the invention provides IL-33 decoy peptides. These peptides can be administered in therapeutically effective amounts to deplete the FLIL33 protein pool to both attenuate the functional effects of intracellular FLIL33 and exhaust the source of the proteolytically mature IL-33 cytokine. In some embodiments the decoy peptide disrupts an association of FLIL33 with an FLIL33 binding partner.

In some embodiments, the invention provides an IL-33 decoy peptide, wherein the decoy peptide comprises a fragment of the N-terminal domain of IL-33 or an IL-33 variant.

As used herein, “IL-33” refers broadly to all or any of the forms of this factor, including its FLIL33 precursor, variants of the MIL33 of varying lengths, as well as various artificial mutants. In some embodiments, these forms include the native C-terminal portion of the molecule (aa 95-270). In some embodiments, they can also include a detection or purification tag. In some embodiments, these forms can contain a C-terminal HA-tag connected by a flexible linker peptide, allowing for easy detection and quantification with anti-HA antibodies (Clerman et al., J Biol Chem, (2017), 292:21653-61: Kopach et al., J Biol Chem, (2014), 289:11829-43; Luzina et al., Am J Respir Cell Mol Biol, 2013, 49:999-1008; Luzina et al., J Immunol, (2012), 189:403-10).

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, for example, an amino acid analog. As used herein, the terms encompass amino acid chains of any length, including full length proteins, wherein the amino acid residues are linked by covalent peptide bonds.

In some embodiments, these decoy peptides comprise fragments of IL-33 or fragments of IL-33 variants, for example, where one or more amino acids are inserted, substituted or deleted. The fragment can be either a contiguous or discontiguous fragment of IL-33 or a IL-33 variant. In some embodiments, the decoy peptides can bind to a receptor or binding partner of FLIL33. In some embodiments, the decoy peptides bind to the receptor or binding partner with similar affinity as the FLIL33. In some embodiments, the decoy peptide binds with higher affinity than FLIL33. In some embodiments, the decoy peptide binds with lower affinity than FLIL33.

In some embodiments, the decoy peptide disrupts an association of FLIL33 with importin 5 (IPO5).

In some embodiments, the decoy peptide binds to the IL-33 binding partner IPO5, and interferes with FLIL33 binding. Without being bound by theory, disruption of the FLIL33 association with IPOS leads to FLIL33 degradation, which depletes FLIL33 and reduces production of MIL33.

In some embodiments, the decoy peptide is a fragment of an IL-33 variant, wherein the variant sequence differs in its ability to bind a receptor or binding partner of FLIL33, relative to the corresponding native, wild-type sequence of FLIL33.

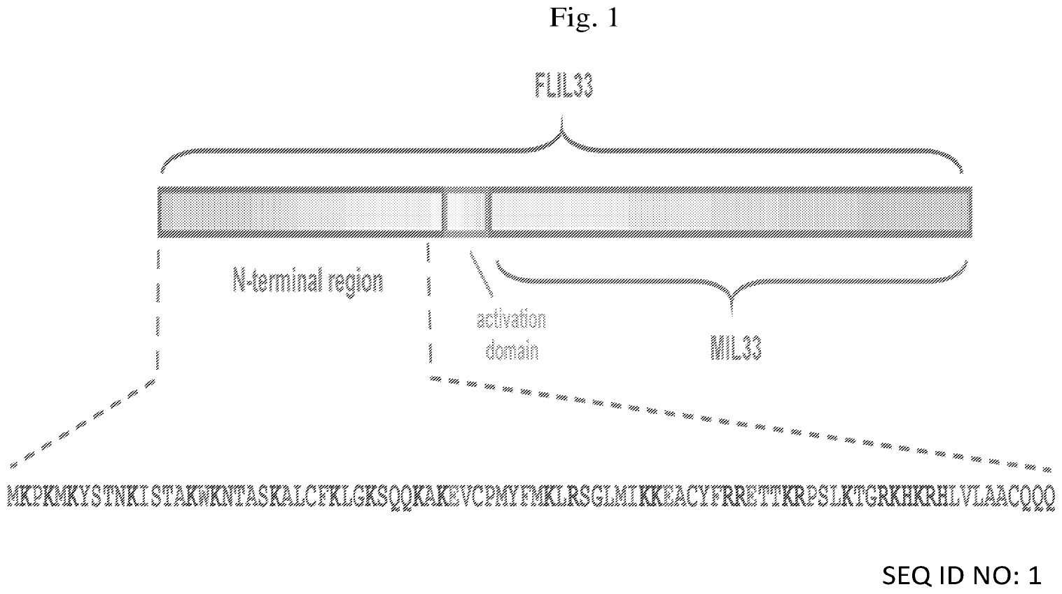

In some embodiments, the fragment of IL-33 or IL-33 variant is derived from the N-terminal region of FLIL33. The domain organization of FILL33, including the amino acid sequence of the N-terminal region, is shown in FIG. 1.

In some embodiments, the amino acid sequence of the N-terminal domain of FLIL33 is SEQ ID NO: 1 (UniProtKB/Swiss-Prot: 095760.1) and is encoded by SEQ ID NO: 2 (NCBI Reference Sequence: NM_033439.4). The amino acid sequence of FLIL33 (UniProtKB/Swiss-Prot: 095760.1) comprises SEQ ID NO:3 and is encoded by SEQ ID NO:4 (NCBI Reference Sequence: NM_033439.4; full length IL-33).

| | |
|---|---|
| SEQ ID | MKPKMKYSTNKISTAKWKNTASKALCFKLGKSQQK |
| NO: 1 | AKEVCPMYFMKLRSGLMIKKEACYFRRETTKRPSL |
| | KTGRKHKRHLVLAACQQQ |
| SEQ ID | ATGAAGCCTAAAATGAAGTATTCAACCAACAAAAT |
| NO: 2 | TTCCACAGCAAAGTGGAAGAACACAGCAAGCAAAG |
| | CCTTGTGTTTCAAGCTGGGAAAATCCCAACAGAAG |
| | GCCAAAGAAGTTTGCCCCATGTACTTTATGAAGCT |
| | CCGCTCTGGCCTTATGATAAAAAAGGAGGCCTGTT |
| | ACTTTAGGAGAGAAACCACCAAAAGGCCTTCACTG |
| | AAAACAGGTAGAAAGCACAAAAGACATCTGGTACT |
| | CGCTGCCTGTCAACAGCAG |
| SEQ ID | MKPKMKYSTNKISTAKWKNTASKALCFKLGKSQQK |
| NO: 3 | AKEVCPMYFMKLRSGLMIKKEACYFRRETTKRPSLK |
| | TGRKHKRHLVLAACQQQSTVECFAFGISGVQKYTRA |
| | LHDSSITGISPITEYLASLSTYNDQSITFALEDESY |
| | EIYVEDLKKDEKKDKVLLSYYESQHPSNESGDGVDG |
| | KMLMVTLSPTKDFWLHANNKEHSVELHKCEKPLPDQ |
| | AFFVLHNMHSNCVSFECKTDPGVFIGVKDNHLALIK |
| | VDSSENLCTENILFKLSET |
| SEQ ID | ATGAAGCCTAAAATGAAGTATTCAACCAACAAA |
| NO: 4 | ATTTCCACAGCAAAGTGGAAGAACACAGCA |
| | AGCAAAGCCTTGTGTTTCAAGCTGGGAAAA |
| | TCCCAACAGAAGGCCAAAGAAGTTTGCCCC |
| | ATGTACTTTATGAAGCTCCGCTCTGGCCTT |
| | ATGATAAAAAAGGAGGCCTGTTACTTTAGG |
| | AGAGAAACCACCAAAAGGCCTTCACTGAAA |
| | ACAGGTAGAAAGCACAAAAGACATCTGGTA |
| | CTCGCTGCCTGTCAACAGCAGTCTACTGTG |
| | GAGTGCTTTGCCTTTGGTATATCAGGGGTC |
| | CAGAAATATACTAGAGCACTTCATGATTCA |
| | AGTATCACAGGAATTTCACCTATTACAGAG |
| | TATCTTGCTTCTCTAAGCACATACAATGAT |
| | CAATCCATTACTTTTGCTTTGGAGGATGAA |
| | AGTTATGAGATATATGTTGAAGACTTGAAA |
| | AAAGATGAAAAGAAAGATAAGGTGTTACTG |
| | AGTTACTATGAGTCTCAACACCCCTCAAAT |
| | GAATCAGGTGACGGTGTTGATGGTAAGATG |
| | TTAATGGTAACCCTGAGTCCTACAAAAGAC |
| | TTCTGGTTGCATGCCAACAACAAGGAACAC |
| | TCTGTGGAGCTCCATAAGTGTGAAAAACCA |
| | CTGCCAGACCAGGCCTTCTTTGTCCTTCAT |
| | AATATGCACTCCAACTGTGTTTCATTTGAA |
| | TGCAAGACTGATCCTGGAGTGTTTATAGGT |
| | GTAAAGGATAATCATCTTGCTCTGATTAAA |
| | GTAGACTCTTCTGAGAATTTGTGTACTGAA |
| | AATATCTTGTTTAAGCTCTCTGAAACTTAG |

In some embodiments, the decoy peptide comprises an amino acid sequence of a fragment of the N-terminal domain of IL-33 or a fragment of the N-terminal domain of an IL-33 variant. In some embodiments, the decoy peptide comprises a sequence selected from one of the following sequences: MKPKMKYSTNKISTAK (2-1) (SEQ ID NO:5); TNKISTAKWKNTASKA (2-2) (SEQ ID NO:6); WKNTASKALCFKLG KS (2-3) (SEQ ID NO:7); LCFKLGKSQQKAKEVC (2-4) (SEQ ID NO:8); QQKAKEVCPMYFMKLR (2-5) (SEQ ID NO:9); PMYFMKLRSGLMIKKE (2-6) (SEQ ID NO:10); SGLMIKKEACYFRRET (2-7) (SEQ ID NO:11); ACYFRRETTKRPSLKT (2-8) (SEQ ID NO:12); TKRPSLKTGRKHKRHL (2-9) (SEQ ID NO:13); and GRKHKRHLVLAACQQQ (2-10) (SEQ ID NO:14).

In some embodiments, the amino acid sequence of the fragment of the IL-33 variant has at least 80% identity to the corresponding sequence of SEQ ID NOS: 1 or 3. In some embodiments, it has at least 85%, 90%, 91%, 92%, 93%, 94%, 95%, 96%, 97%, 98% or 99% identity to the corresponding sequence(s) of SEQ ID NOS: 1 or 3.

The fragment can comprise contiguous or discontiguous fragments of IL-33 or IL-33 variants. In some embodiments, the decoy peptide comprises an amino acid sequence that is at least about 80%, at least about 85%, at least about 90%, 91%, 92%, 93%, 94%, 95%, 96%, 97%, 98%, or at least about 99% identical to one of SEQ ID NOS: 5-14.

A fragment is a polypeptide having an amino acid sequence that entirely is the same as part but not all of the amino acid sequence of the aforementioned IL-33 polypeptide or an IL-33 variant. In some embodiments, the fragment is 10-50 amino acids. In some embodiments, the fragment is 15-30 amino acids. In some embodiments, the fragment is 15-20 amino acids.

In some embodiments, a fragment may constitute no more than about 20 contiguous or discontiguous amino acids of an IL-33 polypeptide (such as SEQ ID NOS: 1 or 3) or an IL-33 variant. In some embodiments, the fragment is not more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous or discontiguous amino acids of an IL-33 polypeptide or an IL-33 variant. In some embodiments, the decoy peptide comprises discontiguous fragments of IL-33. In some embodiments the various fragments can be jointed together by a linker, which can include a chemical conjugation, or one or more amino acids. In some embodiments, the linker can comprise a peptide linker, such as (G3S) 3.

In some embodiments, fragments are characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, high antigenic index regions, or functional domains.

Variants of IL-33 include polypeptides having an amino acid sequence at least about 80%, at least about 85%, at least about 90%, 91%, 92%, 93%, 94%, 95%, 96%, 97%, 98%, or at least about 99% identical to that of SEQ ID NOS: 1 or 3 or fragments thereof with at least about 80%, at least about 85%, at least about 90%, 91%, 92%, 93%, 94%, 95%, 96%, 97%, 98%, or at least about 99% identity to the corresponding fragment of SEQ ID NOS: 1 or 3. In some embodiments, the variants are those that vary from the reference by conservative amino acid substitutions, i.e., those that substitute a residue with another of like characteristics. Typical substitutions are among Ala, Val, Leu and Ile; among Ser and Thr; among the acidic residues Asp and Glu: among Asn and Gln; and among the basic residues Lys and Arg, or aromatic residues Phe and Tyr. In some embodiments, the polypeptides are derivatives in which 1, 2, 3, 4, 5, 6, 7, 8, 9, 10 or more amino acids are substituted, deleted, or added in any combination.

In some embodiments, the decoy peptide is fused to an amino acid sequence that enables entry of the decoy peptide into the cell. In some embodiments, the decoy peptide is a fusion protein comprising i) an amino acid sequence of a fragment of IL-33 or a fragment of an IL-33 variant and ii) an amino acid sequence that enables the peptide to enter a cell. In some embodiments, the decoy peptide comprises an amino acid sequence of a fragment of the N-terminal domain of IL-33 or a fragment of the N-terminal domain of an IL-33 variant. Amino acid sequences enabling cell entry can be broadly, and interchangeably referred to herein as cell penetrating peptides (CPP) or protein transduction domains (PTD).

The CPP or PTD sequence facilitates uptake of the encoded peptide by cells, thereby facilitating the peptide's therapeutic activity when administered to a subject. The CPP or PTD is not limiting and is described above. In some embodiments, the PTD sequence comprises any one of SEQ ID NOS: 15-61.

PTDs or CPPs are short modular motifs, which, when attached to heterologous proteins, can transfer proteins across cell membranes (Guidotti et al., Trends Pharmacol Sci, (2017), 38:406-24: Dinca et al., Int J Mol Sci, (2016), 17:263; Copolovici et al., ACS Nano, (2014), 8:1972-94; Couture et al., J Biol Chem, (2012), 287:24641-8). These short motifs, generally rich in positively charged amino acids, permit transfer of proteins across plasma membrane, without requiring any receptors for their internalization (Guidotti et al., Trends Pharmacol Sci, (2017), 38:406-24: Dinca et al., Int J Mol Sci, (2016), 17:263). Viral and cellular proteins-such as the HIV-TAT, herpes simplex viral VP22, the homeodomain protein antennapedia, lactoferrin and fibroblast growth factor contain such domains, which can be modularly attached to other proteins. The CPPs fused to the decoy peptides enable the peptides to enter cells and to selectively interfere with, through the decoy peptide portion, with the target protein's function and fate by competing with their natural intracellular targets for binding partners.

In some embodiments, the CPP comprises a segment of antennapedia protein from Drosophila termed penetratin and having the sequence of SEQ ID NO:15 (RQIKIWFQNRRMKWKK). In some embodiments, the CPP comprises a segment from the HIV TAT protein and having the sequence of SEQ ID NO:16 (YGRKKRRQRRR). In some embodiments, the CPPs can be C-terminally amidated to increase peptide stability.

The CPP or PTD sequence is not limiting, provided it encodes a peptide sequence that enhances uptake of a functional polypeptide by cells. In some embodiments, the PTD comprises RRRRRRRRRPSASYPYDVPDYA (SEQ ID NO: 17) and is encoded by SEQ ID NO:62 (AGACGAAGGCGCAGACGGAGGCGTAGACCGTCTGCCAGCTATCCATAC GACGTGCCTGACTACGCG). In some embodiments, the PTD nucleic acid sequence comprises a nucleic acid encoding one or more variants of TAT protein from HIV selected from GRKKRRQRRR (SEQ ID NO:18), SEQ ID NO:16 (YGRKKRRQRRR), or GRKKRRQ (SEQ ID NO:19). SEQ ID NO:18 is encoded by SEQ ID NO:63 (GGCCGTAAAAAACGCCGTCAACGCCGCCGT). SEQ ID NO: 16 is encoded by SEQ ID NO:64 (TATGGCCGTAAAAAACGCCGTCAACGCCGCCGT). SEQ ID NO:19 is encoded by SEQ ID NO:65 (GGCCGTAAAAAACGCCGTCAA). Alternate forms of TAT can also be used.

Non-limiting examples of PTDs which can be used in the present invention are shown in the Table below.

### Protein Transduction Domain Sequences

| PROTEIN TRANSDUCTION DOMAINS | SEQ ID NO: |
|---|---|
| RQIKIWFQNRRMKWKK | 15 |
| YGRKKRRQRRR | 16 |
| RRRRRRRRRPSASYPYDVPDYA | 17 |
| GRKKRRQRRR | 18 |
| GRKKRRQ | 19 |
| RRMKWKK | 20 |
| RRWRRWWRRWWRRWRR | 21 |
| RGGRLSYSRRRFSTSTGR | 22 |
| RKKRRQRRR | 23 |
| YARAAARQARA | 24 |
| RRRRRRRR | 25 |
| KKKKKKKK | 26 |
| GWTLNSAGYLLGKINLKALAALAKXIL | 27 |
| SRRHHCRSKAKRSRHH | 28 |
| NRARRNRRRVR | 29 |
| RQLRIAGRRLRGRSR | 30 |
| KLIKGRTPIKFGK | 31 |
| RRIPNRRPRR | 32 |
| KLALKLALKALKAALKLA | 33 |
| KLAKLAKKLAKLAK | 34 |
| GALFLGFLGAAGSTNGAWSQPKKKRKV | 35 |
| KETWWETWWTEWSQPKKKRKV | 36 |
| LKKLLKKLLKKLLKKLLKKL | 37 |
| QAATATRGRSAASRPTERPRAPARSASRPRRP | 38 |
| VE | |
| MGLGLHLLVLAAALQGAKSKRKV | 39 |
| AAVALLPAVLLALLAPAAANYKKPKL | 40 |
| MANLGYWLLALFVTMWTDVGLCKKRPKP | 41 |
| LGTYTQDFNKFHTFPQTAIGVGAP | 42 |
| DPKGDPKGVTVTVTVTVTGKGDPXPD | 43 |
| PPPPPPPPPPPPPP | 44 |
| VRLPPPVRLPPPVRLPPP | 45 |
| PRPLPPPRPG | 46 |
| SVRRRPRPPYLPRPRPPPFFPPRLPPRIPP | 47 |
| TRSSRAGLQFPVGRVHRLLRK | 48 |
| GIGKFLHSAKKFGKAFVGEIMNS | 49 |
| KWKLFKKIEKVGQNIRDGIIKAGPAVAVVGQAT | 50 |
| QIAK | |
| ALWMTLLKKVLKAAAKAALNAVLVGANA | 51 |
| GIGAVLKVLTTGLPALISWIKRKRQQ | 52 |
| INLKALAALAKKIL | 53 |
| GFFALIPKIISSPLPKTLLSAVGSALGGSGGQE | 54 |
| LAKWALKQGFAKLKS | 55 |
| SMAQDIISTIGDLVKWIIQTVNXFTKK | 56 |
| LLGDFFRKSKEKIGKEFKRIVQRIKQRIKD | 57 |
| FLANLVPRTES | |
| PAWRKAFRWAWRMLKKAA | 58 |
| KLKLKLKLKLKLKLKLKL | 59 |
| LLILLRRRIRKQANAHSK | 60 |
| GALFLGWLGAAGSTMGAKKKRKV | 61 |

In some embodiments, the decoy peptide comprises SEQ ID NO:9 or 11, fused to a cell penetrating peptide selected from any of SEQ ID NOS: 15-61. In some embodiments, the decoy peptide is fused to SEQ ID NO:16. In some embodiments, the decoy peptide fused to a cell penetrating peptide comprises SEQ ID NO:66. In some embodiments, the decoy peptide fused to a cell penetrating peptide comprises SEQ ID NO:67.

In some embodiments, the IL-33 decoy peptide comprises SEQ ID NO:9 or SEQ ID NO:66. In some embodiments, the IL-33 decoy peptide comprises SEQ ID NO: 11 or SEQ ID NO:67.

In some embodiments, a linker may be used to connect one or more PTDs and the decoy peptide. In some embodiments, the linker is a peptide linker, such as (GsS) 3, to connect the CPP and decoy peptide.

In some embodiments, the PTD is fused or linked in frame to the N-terminal and/or C-terminal end of the decoy peptide. In some embodiments, the decoy peptide sequences are located downstream from the PTD sequence, i.e., the PTD sequence is N-terminal to the decoy peptide sequence.

The peptides comprising IL-33 decoy peptides can be prepared in any suitable manner. Such polypeptides include recombinantly produced polypeptides, synthetically produced polypeptides, or polypeptides produced by a combination of these methods. Means for preparing such polypeptides are well understood in the art.

In some embodiments, the decoy peptides or fusion peptide nucleic acid sequence has been optimized for expression in alternative host organisms (e.g., non-human). Although as described above, the genetic code is degenerate, so frequently one amino acid may be coded for by two or more nucleotide codons. Thus, multiple nucleic acid sequences may encode one amino acid sequence. Although this creates identical proteins, the nucleic acids themselves are distinct, and can have other distinct properties. As described herein, one aspect of the choice of codon usage can be (but is not limited to) the ability to express a peptide in a non-native cells (e.g., a human protein in bacteria or yeast), or the level of expression in such cells. In order to obtain enough peptide for purification, testing, and use in in vitro assays, in animal models, and eventually in clinical development, efficient protein expression in non-human systems is needed.

In some embodiments, the decoy peptides can further include a one or more of an epitope tag or a purification tag. The term “epitope tag” as used herein refers to peptide sequences that are recognized and bound by the variable region of an antibody or fragment. In some embodiments, the epitope tag is not part of the native protein. In some embodiments, the epitope tag is removable. In some embodiments, the epitope tag is not intrinsic to the protein's native biological activity. Examples of epitope tags include, but are not limited to Myc, HA and FLAG.

The term “purification tag” as used herein refers to peptide sequences that facilitate the purification of the protein, but are generally not necessary for the protein's biological activity. In some embodiments, purification tags may be removed following protein purification. Examples of purification tags include, but are not limited to glutathione S-transferase (GST) or 6x-histidine (H6).

In some embodiments, the epitope tag is selected from Myc. HA and FLAG and combinations thereof. In some embodiments, the purification tag is one or more of glutathione-S-transferase (GST) or 6x-histidine (H6).

In some embodiments, the decoy peptides also comprise a cleavage site for a protease. In some embodiments, the cleavage site is a enterokinase target sequence, located downstream or upstream from one or more epitope and/or purification tags.

### Nucleic Acids

In some embodiments, the invention provides a nucleic acid molecule comprising a nucleotide sequence encoding a decoy peptide that is an IL-33 peptide mimetic.

In some embodiments, a nucleotide sequence encoding the decoy peptide may be derived from genomic DNA, i.e., cloned directly from the genome of a particular organism. In some embodiments, however, the nucleic acid would comprise complementary DNA (cDNA). The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression. In some embodiments, the nucleic acids are mRNAs that can be delivered therapeutically to cells.

In some embodiments, the nucleic acids of the decoy peptides correspond to or are highly similar to nucleic acids that encode FLIL33, particularly, the N-terminal region.

The organismal source of IL-33 nucleic acid sequence is not limiting. In some embodiments, the decoy peptide nucleic acid sequence is derived from a mammal. In some embodiments, the decoy peptide nucleic acid sequence is of human origin. In some embodiments, the decoy peptide nucleic acid sequence is from dog, cat, horse, mouse, rat, guinea pig, sheep, cow, pig, monkey, or ape.

In some embodiments, the nucleic acid is of human origin. In some embodiments, N-terminal FLIL33 is encoded by SEQ ID NO:2 and FLIL33 is encoded by SEQ ID NO:4.

The nucleic acid molecules may be produced using recombinant DNA technology (e.g., polymerase chain reaction (PCR) amplification, cloning) or chemical synthesis. Decoy peptide nucleic acids include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect (e.g., inhibition of IL-33 activity when administered to subjects).

In some embodiments, a particular nucleotide sequence encoding a decoy peptide corresponding to a fragment of IL-33 polypeptide may be identical over its entire length to a coding sequence in SEQ ID NOS: 2 or 4. In some embodiments, a particular nucleotide sequence encoding a decoy peptide may be encoded by sequences other than those found in SEQ ID NOS: 2 or 4 due to degeneracy in the genetic code or variation in codon usage.

In some embodiments, the nucleic acid sequence of the decoy peptide contains a nucleotide sequence that is highly identical, at least 90% identical, with a nucleotide sequence encoding the corresponding IL-33 fragment peptide. In some embodiments, the nucleic acid sequence of the decoy peptide comprises a nucleotide sequence that is at least 60%, 65%, 70%, 75%, 80%, 85%, 90%, 91%, 92%, 93%, 94%, 95%, 96%, 97%, 98% 99% or 100% identical with the corresponding nucleotide sequence set forth in SEQ ID NOS: 2 or 4.

When a polynucleotide of the invention is used for the recombinant production of decoy peptide, the polynucleotide may include the coding sequence for the decoy peptide by itself: the coding sequence for the decoy peptide in reading frame with other coding sequences, such as those encoding a leader or secretory sequence, a pre- or pro or prepro-protein sequence, or other fusion peptide portions. The polynucleotide may also contain non-coding 5′ and 3′ sequences, such as transcribed, non-translated sequences, splicing and polyadenylation signals, ribosome binding sites and sequences that stabilize mRNA. In a preferred embodiment, the nucleotide sequence comprises a nucleotide sequence encoding an amino acid sequence that enables entry into cells (e.g., CPP or PTD).

Conventional means utilizing known computer programs such as the BestFit program (Wisconsin Sequence Analysis Package, Version 10 for Unix, Genetics Computer Group, University Research Park, 575 Science Drive, Madison, Wis. 53711) may be utilized to determine if a particular nucleic acid molecule is at least 90%, 91%, 92%, 93%, 94%, 95%, 96%, 97%, 98% or 99% identical.

### Vectors, Host Cells, and Recombinant Expression

The present invention also relates to vectors that comprise the nucleic acids of the present invention, including cloning vectors and expression vectors, host cells which are genetically engineered with vectors of the invention and methods for the production of polypeptides of the invention by recombinant techniques. Cell-free translation systems can also be employed to produce such proteins using RNAs derived from the DNA constructs of the invention.

Representative examples of appropriate hosts include bacterial cells, such as streptococci, staphylococci, Escherichia coli, Streptomyces and Bacillus subtilis; fungal cells, such as yeast and Aspergillus: insect cells such as Drosophila S2 and Spodoptera Sf9; mammalian cells such as CHO. COS. HeLa. C127, 3T3. BHK. HEK-293 and Bowes melanoma. A great variety of expression systems can be used, including DNA or RNA vectors.

In other embodiments, this invention provides an isolated nucleic acid molecules of the invention operably linked to a heterologous promoter. The invention further provides an isolated nucleic acid molecule operably linked to a heterologous promoter, wherein said isolated nucleic acid molecule is capable of expressing a fusion protein comprising an IL-33 decoy peptide and an amino acid sequence capable of enabling entry of the peptide into cells when used to transform an appropriate host cell.

Methods for the production of polypeptides of the invention including culturing a host cells transfected with one or more of the vectors of the present invention under conditions promoting expression of the polypeptide encoded by the vector, and isolating the polypeptide so expressed from the cell culture.

Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC 2.0 from INVITROGEN and BACPACK baculovirus expression system from CLONTECH.

Other examples of expression systems include COMPLETE CONTROL Inducible Mammalian Expression System from STRATAGENE, which involves a synthetic ecdysone-inducible receptor, or its pET Expression System, an E. coli expression system. Another example of an inducible expression system is available from INVITROGEN, which carries the T-REX (tetracycline-regulated expression) System, an inducible mammalian expression system that uses the full-length CMV promoter. INVITROGEN also provides a yeast expression system called the Pichia methanolica Expression System, which is designed for high-level production of recombinant proteins in the methylotrophic yeast P. methanolica. One of skill in the art would know how to manipulate a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment involves the use of gene transfer to immortalize cells for the production of proteins. The nucleic acid for the protein of interest may be transferred as described above into appropriate host cells followed by culture of cells under the appropriate conditions.

Examples of useful mammalian host cell lines are Vero and HeLa cells and cell lines of Chinese hamster ovary, W138, BHK, COS-7, HEK-293, HepG2, NIH3T3, RIN and MDCK cells. In addition, a host cell clone may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate: gpt, that confers resistance to mycophenolic acid: neo, that confers resistance to the aminoglycoside G418; and hygro, that confers resistance to hygromycin.

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes (e.g., bacteria or yeast), depending upon whether the desired result is replication of the vector or expression of part or all of the vector-encoded nucleic acid sequences. Numerous cell lines and cultures are available for use as a host cell, and they can be obtained through the American Type Culture Collection (ATCC). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5a, JM109, and KCB, as well as a number of commercially available bacterial hosts such as SURE Competent Cells and SOLOPACK Gold Cells (STRATAGENE, La Jolla). Alternatively, bacterial cells such as E. coli LE392 could be used as host cells for phage viruses.

Examples of eukaryotic host cells for replication and/or expression of a vector include HeLa, NIH3T3, Jurkat, HEK-293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Viral Vectors/Gene Delivery Systems

In some embodiments, the invention provides a viral vector encoding one or more IL-33 decoy peptides. In some embodiments, the IL-33 decoy peptide is fused to an epitope tag. The epitope tag is not limiting, and in some embodiments is selected from the group consisting of Myc, FLAG, hemagglutinin (HA) and/or combinations thereof.

The viral vector is not limiting. In some embodiments, the viral vector will typically comprise a highly attenuated, non-replicative virus. Viral vectors include, but are not limited to, DNA viral vectors such as those based on adenoviruses, herpes simplex virus, avian viruses, such as Newcastle disease virus, poxviruses such as vaccinia virus, and parvoviruses, including adeno-associated virus; and RNA viral vectors, including, but not limited to, the retroviral vectors. Vaccinia vectors and methods useful in immunization protocols are described in U.S. Pat. No. 4,722,848. Retroviral vectors include murine leukemia virus, and lentiviruses such as human immunodeficiency virus. Naldini et al. (1996) Science 272:263-267. Replication-defective retroviral vectors harboring a nucleotide sequence of interest as part of the retroviral genome can be used. Such vectors have been described in detail. (Miller et al. (1990) Mol. Cell. Biol. 10:4239; Kolberg, R. (1992) J. NIH Res. 4:43; Cometta et al. (1991) Hum. Gene Therapy 2:215).

Adenovirus and adeno-associated virus vectors useful in the invention may be produced according to methods already taught in the art. (See, e.g., Karlsson et al. (1986) EMBO 5:2377; Carter (1992) Current Opinion in Biotechnology 3:533-539; Muzcyzka (1992) Current Top. Microbiol. Immunol. 158:97-129; Gene Targeting: A Practical Approach (1992) ed. A. L. Joyner. Oxford University Press, NY). Several different approaches are feasible.

Alpha virus vectors, such as Venezuelan Equine Encephalitis (VEE) virus, Semliki Forest virus (SFV) and Sindbis virus vectors, can be used for efficient gene delivery. Replication-deficient vectors are available. Such vectors can be administered through any of a variety of means known in the art, such as, for example, intranasally or intratumorally. See Lundstrom, Curr. Gene Ther. 2001 1:19-29.

Additional literature describing viral vectors which could be used in the methods of the present invention include the following: Horwitz, M. S., Adenoviridae and Their Replication, in Fields, B., et al. (eds.) Virology. Vol. 2, Raven Press New York, pp. 1679-1721, 1990); Graham, F. et al., pp. 109-128 in Methods in Molecular Biology, Vol. 7: Gene Transfer and Expression Protocols, Murray, E. (ed.), Humana Press, Clifton, N.J. (1991); Miller, et al. (1995) FASEB Journal 9:190-199. Schreier (1994) Pharmaceutica Acta Helvetiae 68:145-159; Schneider and French (1993) Circulation 88:1937-1942: Curiel, et al. (1992) Human Gene Therapy 3:147-154: WO 95/00655: WO 95/16772: WO 95/23867; WO 94/26914: WO 95/02697 (Jan. 26, 1995); and WO 95/25071.

In some embodiments, the viral vector is a retrovirus/lentivirus, adenovirus, adeno-associated virus, alpha virus, vaccinia virus or a herpes simplex virus. In some embodiments, the viral vector is a lentiviral vector.

### Therapeutic Methods

In some embodiments, the invention provides a method of treating and/or preventing a disease or condition in a subject that is modulated by IL-33, comprising administering to the subject an effective amount of a decoy peptide as described herein. In preferred embodiments, the decoy peptide comprises an amino acid sequence that enables the peptide to enter cells.

In some embodiments, the invention provides a method of treating and/or preventing a disease or condition in a subject that is modulated by IL-33, comprising administering to the subject an effective amount of a viral vector encoding a decoy peptide as described herein.

In some embodiments, the invention provides a method of treating and/or preventing a disease or condition in a subject that is modulated by IL-33, comprising administering to the subject an effective amount of a nucleic acid encoding a decoy peptide as described herein. In some embodiments, the nucleic acid is mRNA.

The disease or condition that is modulated by IL-33 is not limiting and can include infections, metabolic disorders, inflammation and fibrosis.

In some embodiments, the disease or condition is characterized by infection. In some embodiments, the invention provides a method of treating and/or preventing infection in a subject, comprising administering to the subject an effective amount of a decoy peptide as described herein. In preferred embodiments, the decoy peptide comprises an amino acid sequence that enables the peptide to enter cells.

In some embodiments, the invention provides a method of treating and/or preventing infection in a subject, comprising administering to the subject an effective amount of a viral vector encoding a decoy peptide as described herein.

In some embodiments, the invention provides a method of treating and/or preventing infection in a subject, comprising administering to the subject an effective amount of a nucleic acid encoding a decoy peptide as described herein. In some embodiments, the nucleic acid is mRNA.

In some embodiments, the infection is caused by a virus. In some embodiments, the viral infection is COVID-19.

In some embodiments, the disease or condition is characterized by inflammation. In some embodiments, the invention provides a method of treating and/or preventing inflammation in a subject, comprising administering to the subject an effective amount of a decoy peptide as described herein. In preferred embodiments, the decoy peptide comprises an amino acid sequence that enables the peptide to enter cells.

In some embodiments, the invention provides a method of treating and/or preventing inflammation in a subject, comprising administering to the subject an effective amount of a viral vector encoding a decoy peptide as described herein.

In some embodiments, the invention provides a method of treating and/or preventing inflammation in a subject, comprising administering to the subject an effective amount of a nucleic acid encoding a decoy peptide as described herein. In some embodiments, the nucleic acid is mRNA.

In some embodiments, the disease or condition is characterized by fibrosis. In some embodiments, the invention provides a method of treating and/or preventing fibrosis in a subject, comprising administering to the subject an effective amount of a decoy peptide as described herein. In preferred embodiments, the decoy peptide comprises an amino acid sequence that enables the peptide to enter cells.

In some embodiments, the invention provides a method of treating and/or preventing fibrosis in a subject, comprising administering to the subject an effective amount of a viral vector encoding a decoy peptide as described herein.

In some embodiments, the invention provides a method of treating and/or preventing fibrosis in a subject, comprising administering to the subject an effective amount of a nucleic acid encoding a decoy peptide as described herein. In some embodiments, the nucleic acid is mRNA.

As used herein, “fibrosis” refers to the formation of excess fibrous connective tissue as a result of the excess deposition of extracellular matrix components, for example collagen. Fibrous connective tissue is characterized by having extracellular matrix (ECM) with a high collagen content. The collagen may be provided in strands or fibers, which may be arranged irregularly or aligned. The ECM of fibrous connective tissue may also include glycosaminoglycans.

As used herein. “excess fibrous connective tissue” refers to an amount of connective tissue at a given location (e.g. a given tissue or organ, or part of a given tissue or organ) which is greater than the amount of connective tissue present at that location in the absence of fibrosis, e.g. under normal, non-pathological conditions. As used herein, “excess deposition of extracellular matrix components” refers to a level of deposition of one or more extracellular matrix components which is greater than the level of deposition in the absence of fibrosis, e.g. under normal, non-pathological conditions.

The cellular and molecular mechanisms of fibrosis are described in Wynn. J. Pathol. (2008) 214 (2): 199-210, and Wynn and Ramalingam, Nature Medicine (2012) 18:1028-1040, which are hereby incorporated by reference in their entirety.

The main cellular effectors of fibrosis are myofibroblasts, which produce a collagen-rich extracellular matrix.

In response to tissue injury, damaged cells and leukocytes produce pro-fibrotic factors such as TGFβ, IL-13 and PDGF, which activate fibroblasts to aSMA-expressing myofibroblasts, and recruit myofibroblasts to the site of injury. Myofibroblasts produce a large amount of extracellular matrix, and are important mediators in aiding contracture and closure of the wound. However, under conditions of persistent infection or during chronic inflammation there can be overactivation and recruitment of myofibroblasts, and thus over-production of extracellular matrix components, resulting in the formation of excess fibrous connective tissue.

In some embodiments, fibrosis may be triggered by pathological conditions, e.g. conditions, infections or disease states that lead to production of pro-fibrotic factors such as TGFβ1. In some embodiments, fibrosis may be caused by physical injury/stimuli, chemical injury/stimuli or environmental injury/stimuli. Physical injury/stimuli may occur during surgery, e.g. iatrogenic causes. Chemical injury/stimuli may include drug induced fibrosis, e.g. following chronic administration of drugs such as bleomycin, cyclophosphamide, amiodarone, procainamide, penicillamine, gold and nitrofurantoin (Daba et al., Saudi Med J 2004 June: 25 (6): 700-6). Environmental injury/stimuli may include exposure to asbestos fibers or silica.

Fibrosis can occur in many tissues of the body. For example, fibrosis can occur in the liver (e.g. cirrhosis), lungs, kidney, heart, blood vessels, eye, skin, pancreas, intestine, brain, and bone marrow. Fibrosis may also occur in multiple organs at once.

In embodiments herein, fibrosis may involve an organ of the gastrointestinal system, e.g., of the liver, small intestine, large intestine, or pancreas. In some embodiments, fibrosis may involve an organ of the respiratory system, e.g., the lungs. In embodiments, fibrosis may involve an organ of the cardiovascular system, e.g., of the heart or blood vessels. In some embodiments, fibrosis may involve the skin. In some embodiments, fibrosis may involve an organ of the nervous system, e.g., the brain. In some embodiments, fibrosis may involve an organ of the urinary system, e.g., the kidneys. In some embodiments, fibrosis may involve an organ of the musculoskeletal system, e.g., muscle tissue.

In some embodiments, the fibrosis is cardiac or myocardial fibrosis, hepatic fibrosis, or renal fibrosis. In some embodiments, cardiac or myocardial fibrosis is associated with dysfunction of the musculature or electrical properties of the heart, or thickening of the walls of valves of the heart. In some embodiments fibrosis is of the atrium and/or ventricles of the heart. Treatment or prevention of atrial or ventricular fibrosis may help reduce risk or onset of atrial fibrillation, ventricular fibrillation, or myocardial infarction.

In some embodiments, hepatic fibrosis is associated with chronic liver disease or liver cirrhosis. In some preferred embodiments renal fibrosis is associated with chronic kidney disease.

Diseases/conditions characterized by fibrosis in accordance with the present invention include but are not limited to: respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma: chronic liver disease, primary biliary cirrhosis (PBC), schistosomal liver disease, liver cirrhosis: cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis. Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts: neurological conditions such as gliosis and Alzheimer's disease: muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, nephrogenic systemic fibrosis and cutis keloid: arthrofibrosis: Dupuytren's contracture; mediastinal fibrosis: retroperitoneal fibrosis: myelofibrosis: Peyronie's disease: adhesive capsulitis: kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease: diseases of the eye such as Grave's ophthalmopathy, epiretinal fibrosis, retinal fibrosis, subretinal fibrosis (e.g. associated with macular degeneration (e.g. wet age-related macular degeneration (AMD)), diabetic retinopathy, glaucoma, comeal fibrosis, post-surgical fibrosis (e.g. of the posterior capsule following cataract surgery, or of the bleb following trabeculectomy for glaucoma), conjunctival fibrosis, subconjunctival fibrosis: arthritis: fibrotic pre-neoplastic and fibrotic neoplastic disease; and fibrosis induced by chemical or environmental insult (e.g. cancer chemotherapy, pesticides radiation/cancer radiotherapy).

It will be appreciated that the many of the diseases/conditions listed above are interrelated. For example, fibrosis of the ventricle may occur post myocardial infarction, and is associated with DCM, HCM and myocarditis.

In particular embodiments, the disease/disorder may be one of pulmonary fibrosis, atrial fibrillation, ventricular fibrillation, hypertrophic cardiomyopathy (HCM), dilated cardiomyopathy (DCM), non-alcoholic steatohepatitis (NASH), cirrhosis, chronic kidney disease, scleroderma, systemic sclerosis, keloid, cystic fibrosis, Chron's disease, post-surgical fibrosis or retinal fibrosis.

Treatment, prevention or alleviation of fibrosis according to the present invention may be of fibrosis that is associated with an upregulation of IL-33, e.g. an upregulation of IL-33 in cells or tissue in which the fibrosis occurs or may occur, or upregulation of extracellular and mature IL-33.

Treatment or alleviation of fibrosis may be effective to prevent progression of the fibrosis, e.g. to prevent worsening of the condition or to slow the rate of development of the fibrosis. In some embodiments treatment or alleviation may lead to an improvement in the fibrosis, e.g. a reduction in the amount of deposited collagen fibers.

Prevention of fibrosis may refer to prevention of a worsening of the condition or prevention of the development of fibrosis, e.g. preventing an early stage fibrosis developing to a later, chronic, stage.

As used herein, “treat” and all its forms and tenses (including, for example, treating, treated, and treatment) refers to therapeutic and prophylactic treatment. In certain aspects of the invention, those in need of treatment include those already with a pathological disease or condition of the invention (including, for example, fibrosis), in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the disease or pathological condition.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition.

The subject to be treated herein is not limiting. In some embodiments, the subject to be treated is a mammal. Mammals that can be treated in accordance with the invention, include, but are not limited to, humans, dogs, cats, horses, mice, rats, guinea pigs, sheep, cows, pigs, monkeys, apes and the like. The term “patient” and “subject” are used interchangeably. In some embodiments, the subject is a human.

The therapeutic agent can be administered one time or more than one time, for example, more than once per day, daily, weekly, monthly, or annually. The duration of treatment is not limiting. The duration of administration of the therapeutic agent can vary for each individual to be treated/administered depending on the individual cases and the diseases or conditions to be treated. In some embodiments, the therapeutic agent can be administered continuously for a period of several days, weeks, months, or years of treatment or can be intermittently administered where the individual is administered the therapeutic agent for a period of time, followed by a period of time where they are not treated, and then a period of time where treatment resumes as needed to treat the disease or condition. For example, in some embodiments, the individual to be treated is administered the therapeutic agent of the invention daily, every other day, every three days, every four days, 2 days per week 3 days per week, 4 days per week, 5 days per week or 7 days per week. In some embodiments, the individual is administered the therapeutic agent for 1 week, 2 weeks, 3 weeks, 4 weeks, 1 month, 2 months, 3 months, 4 months, 5 months, 6 months, 7 months, 8 months, 9 months, 10 months, 11 months, 1 year or longer.

In some embodiments, the administration of the decoy peptides, nucleic acids or viral vectors decrease the levels of mature IL-33 in the tissues of the subject. In some embodiments, the levels of mature IL-33 decrease by about 10%, about 20%, about 30%, about 40%, about 50%, about 60%, about 70%, about 80%, about 90%, or about 100% over untreated levels.

In some embodiments, the administration of the decoy peptides, nucleic acids or viral vectors decrease the levels of FLIL33 in the tissues of the subject. In some embodiments, the levels of FLIL33 decrease by about 10%, about 20%, about 30%, about 40%, about 50%, about 60%, about 70%, about 80%, about 90%, or about 100% over untreated levels.

In some embodiments, the subject is administered one or more additional therapeutic agents. In some embodiments, the one or more additional therapeutic agents are those commonly used to treat fibrosis, inflammation or infection.

In some embodiments, the subject is administered decoy peptides, nucleic acids or viral vectors as described herein in combination an anti-inflammatory drug. In some embodiments, the anti-inflammatory drug is a non-steroidal anti-inflammatory drug (NSAID). In some embodiments, anti-inflammatory drug is selected from the group consisting of Antazoline, Balsalazide, Beclometasone, Betamethasone, Budesonide, Celecoxib, Colchicine, Deflazacort, Dexamethasone, Dexibuprofen, Diclofenac, Etanercept, Etodolac, Felbinac, Fenoprofen, Flumetasone, Fluorometholone, Flurbiprofen, Flurbiprofen, Fluticasone, Gentamicin, Hydrocortisone, Ibuprofen, Indometacin, Ketoprofen, Loteprednol, Mefenamic acid, Meloxicam, Mesalazine, Methylprednisolone, Mometasone, Nabumetone, Naproxen, Nepafenac, Olsalazine, Prednisolone, Rimexolone, Sulfasalazine, Sulindac, Tenoxicam, Tiaprofenic acid, Triamcinolone and combinations thereof.

In some embodiments, the subject is administered an effective amount of a combination of viral vector and decoy peptide of the invention.

In some embodiments, the subject is not administered another therapeutic agent and is administered a composition consisting of or consisting essentially of a decoy peptide, viral vector, or nucleic acid of the invention.

### Pharmaceutical Compositions

Where clinical applications are contemplated, it will be necessary to prepare pharmaceutical compositions in a form appropriate for the intended application. Generally, this will entail preparing compositions that are suitable for administration to a subject, e.g., essentially free of pyrogens, as well as other impurities that could be harmful to humans or animals.

In some embodiments, the invention provides pharmaceutical compositions comprising effective amounts of an IL-33 decoy peptide, a nucleic acid encoding an IL-33 decoy peptide, or a viral vector encoding an IL-33 decoy peptide as described herein which are capable of treating of one or more diseases or conditions described herein.

In some embodiments, the nucleic acids can be complexed or encapsulated. For example, the nucleic acids may be complexed or encapsulated in lipidoids, liposomes, lipoplexes, or nanoparticles, such as lipid nanoparticles. See, e.g, U.S. Pat. No. 9,254,311, which is incorporated by reference. In certain embodiments, the nucleic acids, the complex or the nanoparticle further comprise one or more targeting moieties. These moieties can be used to target delivery in vivo to certain organs, tissues or cells.

In some embodiments, the composition comprises appropriate salts and/or buffers to render delivery vectors or peptides stable and allow for uptake by target cells. In some embodiments, compositions comprising a viral vector, nucleic acid or peptide is dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors fusion proteins of the present technology, its use in therapeutic compositions is contemplated. Supplementary active ingredients also can be incorporated into the compositions.

The active compositions of the present technology may include classic pharmaceutical preparations. Administration of these compositions according to the present technology will be via any common route so long as the target tissue is available via that route. Such routes of administration may include oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrastemal, and transdermal), nasal, buccal, urethral, rectal, vaginal, mucosal, dermal, or topical (including dermal, buccal, and sublingual). Alternatively, administration may be by orthotopic, intradermal, subcutaneous, intramuscular, intraperitoneal or intravenous injection. Such compositions would normally be administered as pharmaceutically acceptable compositions. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated.

In some embodiments, compositions which are dispersions can also be prepared, e.g., in glycerol, liquid polyethylene glycols, and mixtures thereof and in oils. Under ordinary conditions of storage and use, these preparations may contain a preservative to prevent the growth of microorganisms.

In some embodiments, pharmaceutical forms suitable for injectable use include sterile aqueous solutions or dispersions and sterile powders for the extemporaneous preparation of sterile injectable solutions or dispersions. In all cases, the form must be sterile and should be fluid to the extent that easy syringability exists. In some embodiments, it must be stable under the conditions of manufacture and storage and must be preserved against the contaminating action of microorganisms, qsuch as bacteria and fungi. In some embodiments, the carrier can be a solvent or dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In some embodiments, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, sterile injectable solutions are prepared by incorporating the viral vector, nucleic acid or peptide in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions can be administered in a variety of dosage forms. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Pharmaceutical compositions suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions.

In one embodiment, compositions may be administered orally in combination with a pharmaceutically acceptable vehicle such as an inert diluent or an edible carrier. Oral compositions may be enclosed in hard or soft shell gelatin capsules, may be compressed into tablets or may be incorporated directly with the food of the patient's diet. The percentage of the composition and preparations may be varied: however, the amount of substance in such therapeutically useful compositions is preferably such that an effective dosage level will be obtained.

Hard capsules containing the compositions may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present technology may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT), zein, shellac, and polysaccharides.

Powdered and granular compositions of a pharmaceutical preparation may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of pharmaceutical compositions which are suitable for oral administration may be prepared, packaged, and sold either in liquid form or in the form of a dry product intended for reconstitution with water or another suitable vehicle prior to use.

A tablet containing one or more active agent compounds described herein may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present technology may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Compositions for parenteral administration include aqueous and non-aqueous sterile injection solutions, which may further contain additional agents, such as antioxidants, buffers, bacteriostats, and solutes, which render the compositions isotonic with the blood of the intended recipient. The compositions may include aqueous and non-aqueous sterile suspensions, which contain suspending agents and thickening agents. Such compositions for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

Compositions for rectal delivery include rectal suppositories, creams, ointments, and liquids. Suppositories may be presented as the active agents in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical compositions may be in any form suitable and readily known in the art for delivery of active agents to the body surface, including dermally, buccally, and sublingually. Typical examples of topical compositions include ointments, creams, gels, pastes, and solutions. Compositions for administration in the mouth include lozenges.

In accordance with these embodiments, oral (topical, mucosal, and/or dermal) delivery materials can also include creams, salves, ointments, patches, liposomes, nanoparticles, microparticles, timed-release formulations and other materials known in the art for delivery to the oral cavity, mucosa, and/or to the skin of a subject for treatment and/or prevention of a condition disclosed herein. Certain embodiments concern the use of a biodegradable oral (topical, mucosal, and/or dermal) patch delivery system or gelatinous material. These compositions can be a liquid formulation or a pharmaceutically acceptable delivery system treated with a formulation of these compositions, and may also include activator/inducers.

The compositions for use in the methods of the present technology may also be administered transdermally, wherein the active agents are incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agents are contained in a layer separate from the adhesive layer. Both types of patches also generally contain a backing layer and a liner that is removed prior to attachment to the recipient's skin. Transdermal drug delivery patches may also be comprised of a reservoir underlying the backing layer that is separated from the skin of the recipient by a semi-permeable membrane and adhesive layer. Transdermal drug delivery may occur through passive diffusion, electrotransport, or iontophoresis.

In certain embodiments, a patch contemplated herein may be a slowly dissolving or a time-released patch. In accordance with these embodiments, a slowly dissolving patch can be an alginate patch. In certain examples, a patch may contain a detectible indicator dye or agent such as a fluorescent agent. In other embodiments, a tag (e.g., detectible tag such as a biotin or fluorescently tagged agent) can be associated with a treatment molecule in order to detect the molecule after delivery to the subject. In certain embodiments, one or more oral delivery patches or other treatment contemplated herein may be administered to a subject three times daily, twice daily, once a day, every other day, weekly, and the like, depending on the need of the subject as assessed by a health professional. Patches contemplated herein may be oral-biodegradable patches or patches for exterior use that may or may not degrade. Patches contemplated herein may be 1 mm, 2 mm, 3 mm, 4 mm to 5 mm in size or more depending on need.

In some embodiments, compositions may include short-term, rapid-onset, rapid-offset, controlled release, sustained release, delayed release, and pulsatile release compositions, providing the compositions achieve administration of the viral vector, nucleic acid or peptides as described herein. See Remington's Pharmaceutical Sciences (18th ed.: Mack Publishing Company, Eaton, Pa., 1990), herein incorporated by reference in its entirety.

In certain embodiments, the compositions disclosed herein can be delivered via a medical device. Such delivery can generally be via any insertable or implantable medical device, including, but not limited to stents, catheters, balloon catheters, shunts, or coils. In one embodiment, the present technology provides medical devices, such as stents, the surface of which is coated with a compound or composition as described herein. The medical device of this technology can be used, for example, in any application for treating, preventing, or otherwise affecting the course of a disease or condition, such as those disclosed herein.

The present invention is further illustrated by the following Examples. These Examples are provided to aid in the understanding of the invention and are not to be construed as a limitation thereof.

Described in the below Examples are studies that form the basis for therapeutic manipulation of IL-33 in diseases or conditions, such as inflammatory fibrotic diseases, through the development of an integrated understanding of the proteolytic maturation and extracellular release of MIL33, intracellular signaling and functioning of FLIL33, and proteolytic stability of the FLIL33 protein pool. The studies of FLIL33 have revealed a wealth of information about its pathobiology and intracellular regulation, such as data related to the molecular control of) the proteasomal degradation of intracellular FLIL33 protein, which is dependent on importin 5 (IPO5).

### EXAMPLES

Example 1. Developing a cell-permeable decoy peptide-based approach to deplete intracellular FLIL33, thereby exhausting the source of MIL33 and simultaneously attenuating the independent effects of FLIL33 and evaluating the in vivo efficacy of such FLIL33 depletion.

We recently reported that FLIL33 binds IPO5 in its N-terminal region (aa 46-56) and that such binding does not affect nuclear shuttling but protects FLIL33 from proteasomal degradation (Clerman et al., J Biol Chem, (2017), 292:21653-61: Kopach et al., J Biol Chem, (2014), 289:11829-43). In this example, we propose to exploit this finding to develop a novel therapy aimed at depleting the levels of FLIL33. Competitive binding of IPOS by CPPs fused with DPs that mimic the aa 46-56 region of FLIL33 is proposed (see Background for CPP-DP-based approach). With less IPOS available for binding to FLIL33, the latter will be exposed to proteasomal degradation. We have considered the risks that 1) a substantial systemic depletion of IL-33 may have undesired effects or 2) be insufficient to attenuate fibrosis. Assuaging the first concern, many groups, including us, have tested germline-deficient mice lacking IL-33 and observed that they demonstrate normal growth, behavior, and lifespan, and manifest no signs of distress, such as ruffled fur, hunched posture, reduced mobility, diarrhea, weight lost, or tachypnea. This is likely because IL-33 is less involved or uninvolved in homeostatic maintenance and more involved in injury- and disease-related regulation. Thus, systemic targeting of IL-33 appears to be safe from a therapeutic standpoint. Allaying the second concern, we developed a tamoxifen (TAM)-inducible mouse model of systemic IL-33 deficiency and tested it, alongside germline-deficient IL-33-knockout (KO, IL33) mice in both chronic and acute models of bleomycin (BLM) injury (FIG. 8). In our inducible model, the IL-33 gene is homozygously floxed (IL33fl/fl). These mice are also transgenic for TAM-activated Cre recombinase, which was inserted into the Gt (ROSA) 26Sor locus, as in one of the parental strains from The Jackson Laboratory (JAX stock 008463), either homozygously (Cre^{tg/tg}) or heterozygously (Cre^{tg/-}). In the latter case, the mice are also heterozygously transgenic for the tdTomato fluorescent protein (tdT^{-/tg}) under the control of the CAG promoter with a floxed STOP, as in one of the parental strains (JAX stock 007914). We induced Cre-based recombination by TAM injection daily in 4 week-old mice for 5 days and compared with oil-injected controls. In IL33^{fl/fl}Cre^{tg/+}tdT^{-/tg} mice, strong whole-body red fluorescence ensues within 72 h after the first injection and persists without fading for the entire lifespan of the mouse (FIG. 9A), indicating the life-long consequences of Cre induction. We then waited for 8 weeks to allow for recovery from possible confounding toxic effects of TAM itself and began inducing the chronic BLM model in 12-week-old mice as described in Example 3 below. Two such experiments have been performed on separate occasions by different technicians, revealing similar results (FIG. 9B, C). Furthermore, not only these inducible IL-33-deficient IL33^{fl/fl}Cre^{tg/tg} mice, but also constitutive IL33^{-/-} mice, were protected from the chronic bleomycin injury-induced elevation in pulmonary collagen (FIG. 9D). Moreover, the levels of collagen were strongly suppressed in response to acute intratracheal BLM injury in TAM-induced IL33^{fl/fl}Cre^{tg/tg} mice (FIG. 9E)

These data indicate that attenuating the levels of IL-33 would be therapeutic in patients with fibrotic diseases. We propose development of a new IL-33 attenuating therapy based on CPP-DP-based strategy. In preliminary tests, we have considered two DPs (Series 1), including the mimic of the IPOS-binding aa 46-56 region of FLIL33 (peptide 1-1) and the mimic of the adjacent aa 60-73 region that does not bind IPOS and thus serves as a control (peptide 1-2). In each peptide 1-1 and 1-2, the CPP penetratin was N-terminally fused to the DP. The final sequences of peptides 1-1 and 1-2 were RQIKIWFQNRRMKWKKFMKLRSGLMIKKEA (SEQ ID NO:69) and ROIKIWFONRRMKWKKFRRETTKRPSLKTG (SEQ ID NO:70), respectively, with the CPP portion shown in italic and the DP portions underlined. Primary fibroblasts were electroporated with FLIL33-encoding and, separately, control non-coding vehicle (NULL) plasmids, and, after 24 h, the medium was replaced with added peptides 1-1 or 1-2. Based on our experience with CPP-DPs, initial concentrations of 20 μM and 40 μM were tested. After an additional 24 h, cells were lysed and the lysates were analyzed for the levels of IL-33 by ELISA. It was immediately obvious that peptide 1-1 was highly toxic to cells, either FLIL33-overexpressing or NULL-transfected, causing rapid (within hours) cell death, whereas the control peptide 1-2 had no effect on cell viability or the expression levels of IL-33. Tests with serial dilutions of peptide 1-1 revealed that it remains toxic in concentrations as low as 2.5 μM, but becomes non-toxic and fails to deplete the levels of FLIL33 at lower concentrations, resulting in a very narrow therapeutic window. The most likely explanation for this effect is based on our previous experience of depleting IPOS with CRISPR-Cas9, AdV-shRNA, and siRNA technologies (Clerman et al., J Biol Chem. (2017). 292:21653-61: Kopach et al., J Biol Chem. (2014). 289:11829-43). We previously observed that a complete or nearly complete depletion of IPO5 using these methodologies quickly leads to cell death, whereas substantial yet incomplete depletion of IPO5 levels abrogates FLIL33 without affecting cell viability. We argue that peptide 1-1 is “too effective” in binding IPO5, thus competing for it not only with FLIL33 but also with other critical molecular partners, and ultimately impeding on cell survival. We then undertook a more rigorous, unbiased approach, based on the following considerations. As stated above in relation to Example 1, there are possible multiple other regions in the N-terminal portion of FLIL33 that control its stability, subcellular localization, secretion, maturation, and functional binding of intracellular molecular partners. Targeting by DPs needs to cover the entire N-terminal part of FLIL33 and be performed in a systematic fashion, and the data integrated with the observations in Examples 1 and 2. The data obtained with peptides 1-1 and 1-2 also suggested the need to tame down the excessive depletion of IPO5, which can be achieved through mutating the decoy peptide, utilizing only a part of the target sequence, using a different CPP, or a combination of these variations. We began by designing Series 2 of overlapping DPs that together cover the entire N-terminus of FLIL33. In all cases, a different CPP (TAT) is fused on the N terminus, based on the possibility that it may decrease the peptide load per cell and thus limit toxicity. All peptides in Series 2 have already been synthesized, and initial cell culture screening tests performed, revealing that peptide 2-5 induced the most pronounced (exceeding 60% of the original levels) depletion of IL-33. We then proceeded to test the capacity of this peptide to deplete the levels of IL-33 in vivo and to determine whether such depletion may be protective, in the therapeutic mode, against fibrosis in the acute BLM injury model. Indeed, peptide 2-5 showed an encouraging therapeutic effect, whereas peptide 2-10 did not. Neither of these two peptides caused any signs of toxicity such as such as ruffled fur, hunched posture, reduced mobility, diarrhea, weight lost, or tachypnea. Thus, peptide 2-5 fused with TAT may be a promising therapeutic agent. However, this and other peptides in this series need to be considered in detail.

Experimental Approach. The approach will be based on our recent discoveries that the intracellular pool of FLIL33 can be depleted through proteasomal degradation and that binding of IPO5 to a short segment in the FLIL33 N-terminal region (KLRSGLMIKKE) (SEQ ID NO:71) protects FLIL33 from being degraded (Clerman et al., J Biol Chem, (2017), 292:21653-61: Kopach et al., J Biol Chem, (2014), 289:11829-43). The derivatives of this sequence will be initially used as a DPs to compete with FLIL33 for IPO5 binding, thus reducing the amount of IPO5 available to protect FLIL33 and allowing its proteasomal degradation. Such CPP-DPs will be tested in primary fibroblast cultures along with control fusion peptides, in which the DP portion will be either scrambled or designed to mimic a segment of FLIL33 that is not involved in controlling its degradation. To account for the possibility of other unknown segments controlling FLIL33's stability in an unbiased fashion, series 2 of overlapping DPs jointly spanning the N-terminal portion of FLIL33 will be tested. There are currently no reliable algorithms to design “perfect” CPP-DPs, and each of these constructs needs to be experimentally tested, as they often require additional modifications to increase their efficiency. If we observe that our CPP-DP constructs fail to efficiently attenuate the intracellular levels of FLIL33, we will redesign the fusion peptides to include a CPP component other than the traditional TAT or penetratin (Kang et al., J Clin Invest, (2017), 127:2541-54; Guidotti et al., Trends Pharmacol Sci, (2017), 38:406-24: Dinca et al., Int J Mol Sci, (2016). 17:263: Copolovici et al., ACS Nano, (2014), 8:1972-94). It is possible that the close proximity of the CPP and DP components of the fusion peptide will interfere with IPO5 binding, in which case we will introduce a neutral peptide linker, such as (G3S) 3, to connect CPP and DP. We may also include multiple copies of the DP in the fusion peptide to enhance IPOS binding. Thus identified CPP-DPs will be additionally validated in HDMEC, HPMEC, HaCaT, and A549 cells. Once the optimal CPP-DP(s) is/are found and optimal concentrations in cell culture are determined, subsequent studies will test them for the ability to attenuate inflammation and fibrosis in the three distinct animal models described in Methods.

### Methods

CPP-DP. We have already designed and initiated the testing of CPP-DPs and their corresponding controls in primary fibroblast cultures. The corresponding peptides targeting mouse FLIL33 expression will be similarly designed based on the substantial homology of FLIL33 protein sequences between humans and mice. Should the need arise depending on the results of initial studies with the peptide constructs, derivative peptides will be designed as stated under the Experimental Approach above. In all cases, C-terminal amidation will be used to increase peptide stability.

Studies in human and mouse cell cultures. Cells will be transfected with recombinant plasmids encoding FLIL33 and, as controls, MIL33 and FLIL37. CPP-DPs and control CPPs will be added to cell culture, and the levels of the overexpressed proteins assessed in cell lysates by ELISA as well as by WB for the HA-tag, which is attached to the C-terminus of all of these proteins. Our experience suggests that their initial testing in cell culture should be started at 20 UM and 40 μM concentrations, followed by titration to reach the lowest concentration at which the levels of FLIL33 are notably depleted.

Mouse models. No model fully recapitulates the entirety of features of human inflammatory fibrotic disorders, and each model has its strengths and weaknesses. Three complementary models will be employed to test the efficacy of CPP-DPs in attenuating the levels of IL-33 as well as decreasing inflammation and fibrosis. We are skilled at studying these animal models as well as in vivo testing of CPP-DPs (Luzina et al., Am J Respir Cell Mol Biol, 2013, 49:999-1008: Pochetuhen et al., Am J Pathol, (2007), 171:428-37; Wyman et al., Am J Physiol Lung Cell Mol Physiol, (2017), 312: L945-L58.: Luzina et al., J Pharmacol Exp Ther, (2015), 355:13-22: Couture et al., J Biol Chem, (2012), 287:24641-8; Javmen et al., J Immunol, (2018), 201:995-1006; Piao et al., Proc Natl Acad Sci USA 2013, 110:19036-41; Piao et al., Cell Rep 2015, 11:1941-52: Piao et al., J Immunol, (2013), 190:2263-72; Toshchakov et al., J Immunol, (2005), 175:494-500; Toshchakov et al., J Immunol, (2007), 178:2655-60; Toshchakov et al., J Immunol, (2011), 186:4819-27).

Model 1. Initial experiments will be performed in FLIL33-overexpressing and control MIL33-overexpressing mice following AdV-mediated gene delivery of precursor and mature IL-33 (Luzina et al., Am J Respir Cell Mol Biol, 2013, 49:999-1008: Luzina et al., J Immunol, (2012), 189:403-10). These two IL-33 forms do not induce overt fibrosis by themselves (without a second insult, e.g., with BLM), but the model is easily developed by a single IT AdV instillation. The elevations in IL-33 levels are rapid and lasting (days 3-21), and the expression levels are pronounced (Luzina et al., Am J Respir Cell Mol Biol, 2013, 49:999-1008; Luzina et al., J Immunol, (2012), 189:403-10). The effects of CPP-DPs that will be examined are changes in IL-33 expression levels and tissue infiltration with inflammatory cells (Luzina et al., Am J Respir Cell Mol Biol, 2013, 49:999-1008; Luzina et al., J Immunol, (2012), 189:403-10). While this model will offer rapid insight into the efficacy of CPP-DPs, the concern is with robust IL-33 expression, which is so substantial that it may be difficult to attenuate with non-toxic CPP-DP doses.

Model 2. Chronic administration of BLM, either with an osmotic pump or repeated intraperitoneal (IP) injections, more accurately models the histological and molecular features of human fibrosis than does acute, locally administered BLM models (Lee et al., Am J Physiol Lung Cell Mol Physiol, (2014), 306: L736-48: Watanabe et al., PLOS One. (2017), 12: e0179917; Luo et al., FASEB J, (2016), 30:874-83: Philip et al., FASEB J, (2017), 31:4745-58; Sun et al., J Clin Invest, (2006), 116:2173-82; Zhou et al., J Immunol, (2011), 186:1097-106). We have successfully adapted both versions of this model and found them to generate similar systemic fibrosis, particularly in the skin and lungs. Alzet osmotic minipumps containing 100 U/kg of BLM or 100 μl of PBS as a control are implanted subcutaneously under the loose skin on the backs of mice for 10 days. After minipump removal, the animals are followed for up to 35 days to assess the fibrotic response in the skin and lungs. Alternatively, mice are injected IP with 0.018 U of BLM or PBS (control) on days 0, 4, 7, 11, 14, 17, 21, and 25 and followed for up to 35 days, with tissue fibrosis detectable as early as day 10. Such utilization of both versions of the model will allow for more reliable, objective results.

Model 3. The limitation of the BLM model is that it is induced by a toxic chemical, which is not the cause of the majority of human fibrotic diseases. Despite the phenotypic similarities between the model and human diseases, the mechanisms are not necessarily similar. Human fibrosis is well known to depend on activation of the TGF-β receptor (TGFBR), and our collaborator. Dr. Sergio Jimenez, has developed a model of systemic fibrosis based on fibroblast-specific, constitutive activation of TGFBR (and Letter of Support) (Wermuth et al., PLOS One, (2018), 13: e0196559). This model, which is referred to as TBRIcaColla2Cre, will be shared with us and utilized similarly to the recently described preclinical trial of a different drug using these mice (Wermuth et al., PLOS One, (2018), 13: e0196559).

Therapeutic regimen of CPP-DP administration. It is currently impossible to predict whether a person will develop a fibrotic disease, and patients commonly present with already established tissue fibrosis. Therefore, CPP-DPs will be tested only in a therapeutic but not a preventive regimen and will be administered systemically by IP injections. CPP-DPs have never been tested in similar models, and there is no precedent that can be relied upon in designing a specific antifibrotic CPP-DP regimen. We found that a 5-day course of daily IP administration of 200 nmol of a CPP-DP is safe and elicits a therapeutic effect in a mouse model of influenza (Piao et al., Cell Rep 2015, 11:1941-52). We will initially utilize this regimen and then adjust it to find a CPP-DP dose that is simultaneously efficacious and non-toxic. Our studies with other CPP-DPs indicate that some of them are efficacious at doses as low as 10 nmol (Couture et al., J Biol Chem, (2012), 287:24641-8; Javmen et al., J Immunol, (2018), 201:995-1006; Piao et al., Proc Natl Acad Sci USA 2013, 110:19036-41; Piao et al., Cell Rep 2015, 11:1941-52; Piao et al., J Immunol, (2013), 190:2263-72: Toshchakov et al., J Immunol, (2005), 175:494-500; Toshchakov et al., J Immunol, (2007), 178:2655-60; Toshchakov et al., J Immunol, (2011), 186:4819-27). The timing of CPP-DP administration may also need to be adjusted. It is possible that in Models 2 and 3, a course of 5 consecutive days of CPP-DP administration may be too short for treatment of characteristic chronic fibrosis, in which case we will extend the treatment period and consider replacing daily injections with drug administration every other day or twice a week. Such adjustments will be guided by the data from the initial experiments.

Experimental readouts and assessment of toxicity. The effects of CPP-DPs will be tested in skin and lung tissues. Tissue homogenates will be tested for changes in the levels of total IL-33 (ELISA, WB) and in total collagen (QuickZyme hydroxyproline assay and WB). Tissue sections will be analyzed for accumulation of inflammatory cells (H&E stain) and fibrotic deposits (trichrome stain). We are skilled in all these assays (Clerman et al., J Biol Chem, (2017), 292:21653-61: Kopach et al., J Biol Chem, (2014), 289:11829-43; Luzina et al., Am J Respir Cell Mol Biol, 2013, 49:999-1008: Luzina et al., J Immunol, (2012), 189:403-10; Luzina et al., Am J Physiol Lung Cell Mol Physiol, (2016), 310: L940-54; Luzina et al., J Leukoc Biol, (2011), 89:763-70; Luzina et al., Immunology, (2011), 132:385-93: Luzina et al., Arthritis Rheum, (2006), 54:2643-55; Pochetuhen et al., Am J Pathol, (2007), 171:428-37; Wyman et al., Am J Physiol Lung Cell Mol Physiol, (2017), 312: L945-L58: Luzina et al., J Pharmacol Exp Ther, (2015), 355: 13-22: Nacu et al., J Immunol. (2008), 180:5036-44: Luzina et al., Arthritis Rheum, (2009), 60:1530-9; Luzina et al., Am J Respir Cell Mol Biol, (2006), 35:298-305; Lee et al., Am J Physiol Lung Cell Mol Physiol, (2014), 306: L736-48; Watanabe et al., PLOS One, (2017), 12: e0179917; Luo et al., FASEB J, (2016), 30:874-83; Philip et al., FASEB J, (2017), 31:4745-58: Sun et al., J Clin Invest, (2006), 116:2173-82; Zhou et al., J Immunol, (2011), 186:1097-106; Wermuth et al., PLOS One, (2018), 13: e0196559). Mice will be monitored daily for signs of toxicity from BLM or CPP-DPs. Based on our experience, mice lose <15% of body weight in response to BLM challenge at the indicated doses. It is expected that treatment with CPP-DPs will minimize the loss of body weight. Mice will be weighed daily, and if they lose >20% of body weight, this will be considered a toxic effect of the inhibitor. Other signs of toxicity include anorexia, dehydration, dyspnea, hunched posture, and ruffled fur. If toxicity develops, the mice will be euthanized to minimize pain and distress.

Sample size and statistical analyses. Mice will be treated with a CPP-DP and a control peptide, with five mice per group. Thus, initial experiments with the regimen proposed above will require 3 models×2 groups×5 mice per group=30 mice. We will test the top two CPP-DPs based on cell culture studies, thus increasing the total minimal required number of mice to 2×30=60. Estimating that at least three different regimens may need to be tested (3×30=90 additional mice), a final total of 60+90=150 mice will be required. This number may be adjusted if additional CPP-DPs need to be tested or regimen modifications become necessary.

Data Analyses and Interpretations. Specific expected and unexpected results of cell culture and in vivo experiments are discussed and interpreted above under the Experimental Approach and Methods. The results will inform future development of anti-inflammatory and antifibrotic therapies based on FLIL33 depletion and, beyond the scope of this project, CPP-DP-based modulation of the functions mapped to other segments of FLIL33.

### Example 2. Interleukin-33 Decoy Peptides Deplete IL-33 In Vivo

Interleukin (IL)-33 acting through its specific cell-surface receptor, ST2 (a.k.a. T1/ST2 and IL1RL1) has been shown to centrally mediate a number of pathophysiological processes. It is therefore not surprising that medicines targeting IL-33 and ST2 are being actively developed and have already demonstrated therapeutic promise in preclinical models as well as clinical trials. Such targeting is achieved either by 1) blocking antibodies, or 2) recombinant cytokine “traps” constructed as fusion proteins between the constant region of IgG and the extracellular domain of the cognate receptor, or even 3) small molecules. Targeting of the IL-33-ST2 axis is likely to be highly efficacious in a variety of diseases. However, not all IL-33-dependent disease processes are controlled by the mature IL-33 cytokine through the IL-33-ST2 axis. Unlike the mature IL-33 (MIL33) cytokine, which is a highly biologically active extracellular mediator, the precursor form, full-length IL-33 (FLIL33), is basally and inducibly expressed, resides mostly in the nucleus of a wide variety of abundant cell types, and modulates inflammatory responses, wound healing, chromatin stability, and transcriptional regulation in a receptor-independent manner. For example, we have shown that elevated FLIL33 promotes pulmonary fibrosis even in the absence of ST2 (PMID 23837438, PMID 36603504). The traditional cytokine-receptor axis-targeting therapies would not be able to reach and neutralize the intranuclear FLIL33 and thus would be less efficacious in diseases that are driven by the ST2-independent effects of IL-33 precursor rather than the ST2-dependent effects of the mature cytokine form. Therapeutic alteration of intracellular targets is technically challenging, but should it be readily achievable, depleting intracellular FLIL33 would provide two benefits. First, it would attenuate the ST2 receptor-independent disease-driving effects of FLIL33. Second, it would deplete the source of extracellular MIL33 which originates from FLIL33 through proteolytic maturation. Thus, a FLIL33-targeting medicine would arguably be therapeutic in a broader spectrum of diseases, including both ST2-independent FLIL33-driven as well as ST2-dependent MIL33-mediated.

We have reported (PMID 29127199) that a segment of FLIL33 represented by amino acids (aa) 46-56 controls interaction with importin-5, and such interaction is critical for preserving the intracellular stability of FLIL33. If this region is deleted or mutated, cell rapidly degrade FLIL33 through a proteasome mechanism (PMID 29127199).

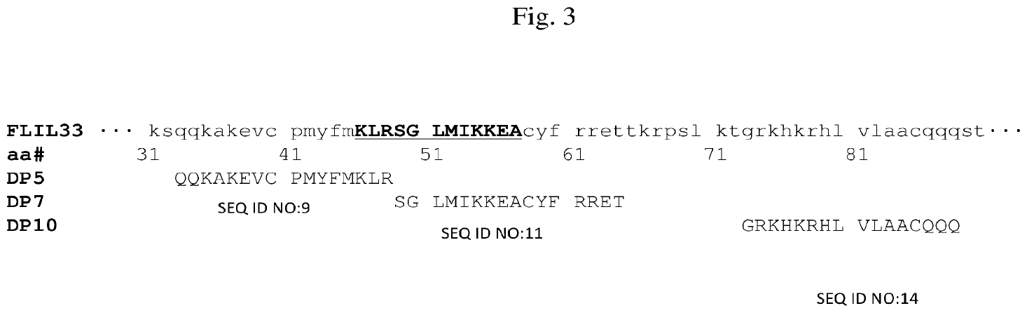

We propose that decoy peptides (DPs) mimicking all or a portion of the importin-5-binding site of FLIL33 may be used to compete with FLIL33 for IPO5 binding. Two of such potential decoy peptides partially overlapping with the importing-5-binding segment of FLIL33, DP5 and DP7, are depicted in FIG. 3. Without protection from importin-5, FLIL33 would then be naturally depleted through the proteasome-dependent mechanism. A peptide mimicking a more distant segment of IL-33 is not expected to compete with FLIL33 for importin-5 binding and thus is not expected to induce depletion of IL-33: such peptide termed DPP10 is also depicted in FIG. 3.

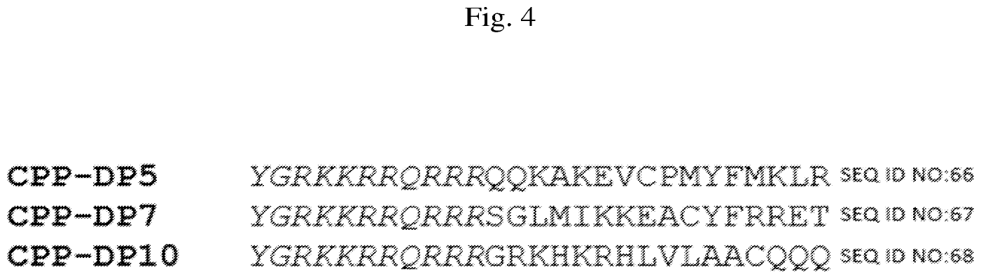

To deliver these DPs inside the target cells, cell-penetrating (also referred to as cell-permeable) peptide (CPP)-based technology is being utilized. We chose a commonly used CPP, a segment from the HIV TAT protein (YGRKKRRQRRR) (SEQ ID NO: 16), to create fusion CPP-DP constructs, including CPP-DP5. CPP-DP7, and CPP-DP10 (FIG. 4)

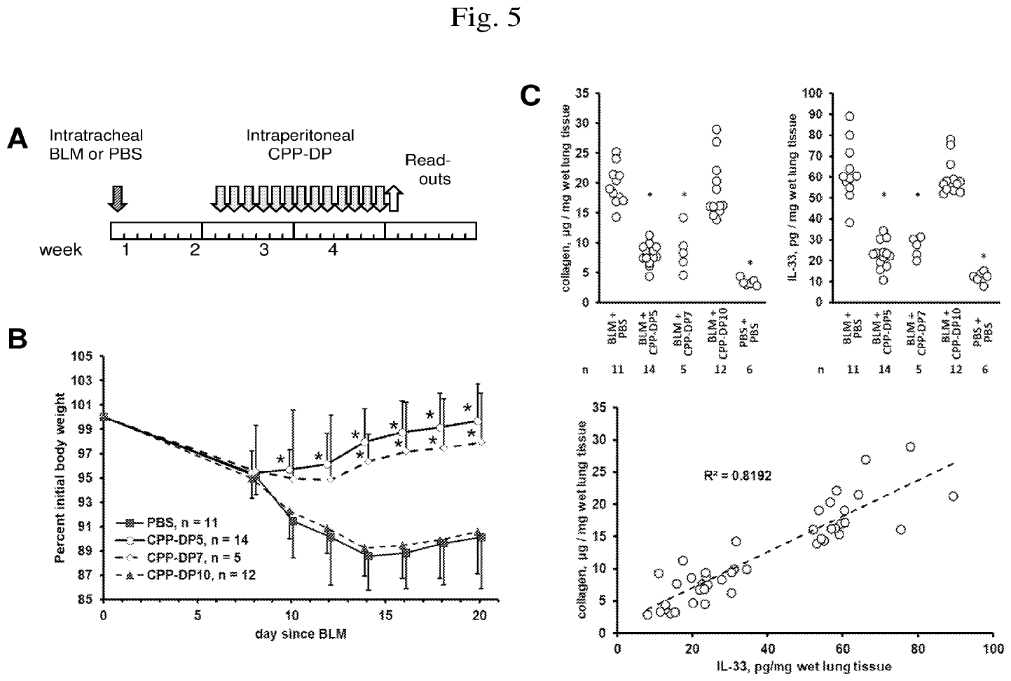

To test the ability of these peptides to deplete the levels of IL-33 in vivo and to assess their ability to modulate the degree of pulmonary fibrosis, these peptides were synthesized, HPLC-purified, and tested in the therapeutic regimen in mice challenged intratracheally with a single dose bleomycin (BLM). In this acute BLM challenge mode, mice received intratracheal 0.08 U of BLM or an equal volume phosphate-buffered saline (PBS) control and allowed to respond to such injury for a week without any intervention. On day 8, intraperitoneal administrations of CPP-DPs once a day were started and continued for 2 weeks. In the end of the 3 weeks, mice have been euthanized, lungs extracted, and pulmonary levels of IL-33 and collagen measured. The experimental outline for these experiments is shown in FIG. 5A.

As expected, mice challenged with BLM and treated with PBS progressively lost body weight, with gradual recovery starting two weeks later. The body weight of BLM-challenged, CPP-DP10-treated mice closely followed the same trend (FIG. 5B). By contrast, mice treated with CPP-DP5 and CPP-DP7 quickly recovered their body weight following the initiation of therapy. BLM-challenged mice treated with CPP-DP5 and CPP-DP7 but not with CPP-DP10 accumulated significantly less collagen (FIG. 5C top left) and IL-33 (FIG. 5C top right) than PBS-treated animals. The collagen and IL-33 values correlated across the experimental groups (FIG. 5C bottom).

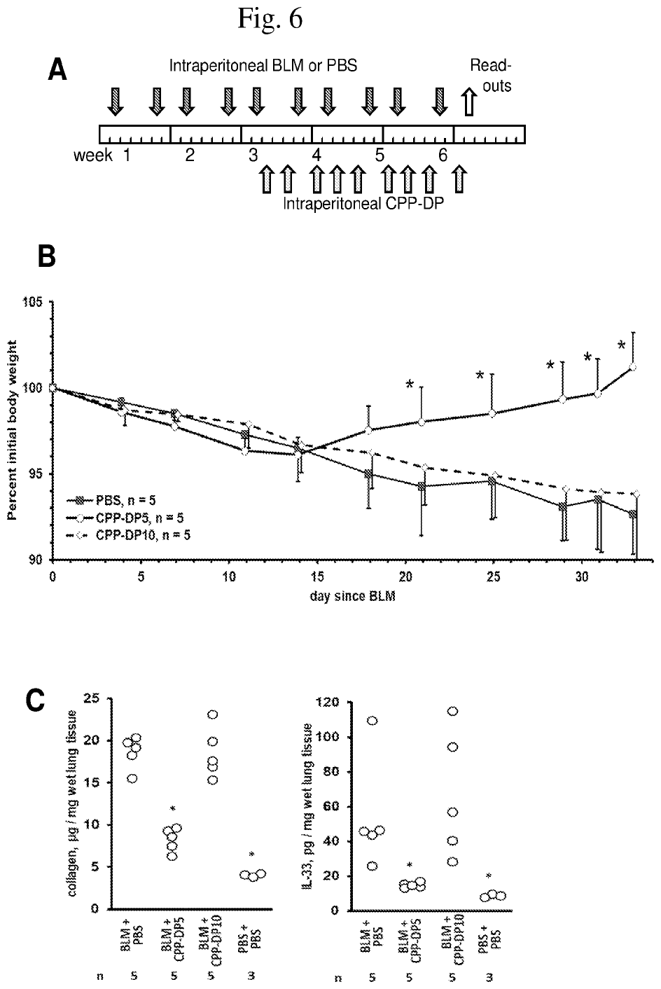

We then considered that such acute model of bleomycin injury, despite its universal use in this field of work, is well known to represent the chronic nature of human pulmonary fibrosis less accurately than the chronic model of systemic BLM delivery. In the chronic model, BLM is injected intraperitoneally twice a week for 33 days, with subsequent assessment of pulmonary changes (FIG. 6A). We challenged mice in this fashion, and, after 14 days such chronic challenge at which point the decline in their total body weight became obvious (FIG. 6B), initiated treatment with CPP-DP5, CPP-DP10, or PBS injected intraperitoneally three times a week (FIG. 6A). Mice treated with CPP-DP5 but not CPP-DP10 or PBS regained their body weight in a time-dependent fashion (FIG. 6B). Of note, such therapy with CPP-DP5 was efficacious despite the continued challenge with repeated administration of BLM (FIG. 6A). Mice treated with CPP-DP5 also accumulated significantly less collagen (FIG. 6C, left) and IL-33 (FIG. 6C, right) than did CPP-DP10-ttreated and PBS-treated controls.

These combined findings support the hypothesis that DPs mimicking the importin-5-binding region of FLIL33 attenuate the levels of IL-33 in vivo, as well as further support the notion that a decrease in IL-33 levels has an antifibrotic effect.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. An IL-33 decoy peptide comprising an amino acid sequence of a fragment of an N-terminal domain of FLIL33 or a fragment of an N-terminal domain of a FLIL33 variant, wherein the decoy peptide disrupts an association of FLIL33 with an FLIL33 binding partner.

2. The IL-33 decoy peptide of claim 1, wherein the decoy peptide further comprises an amino acid sequence that enables the peptide to enter a cell.

3. The IL-33 decoy peptide of claim 1, wherein the decoy peptide comprises an amino acid sequence of a fragment of the N-terminal domain of FLIL33.

4. The IL-33 decoy peptide of claim 3, wherein the decoy peptide disrupts an association of FLIL33 with IPO5.

5. The IL-33 decoy peptide of claim 3, wherein the N-terminal domain of FLIL33 comprises SEQ ID NO:1.

6. The IL-33 decoy peptide of claim 1, wherein the peptide comprises an amino acid sequence selected from the group consisting of any one of SEQ ID NOS: 5-14.

7. The IL-33 decoy peptide of claim 1, wherein the fragment of FLIL33 is a contiguous fragment.

8. The IL-33 decoy peptide of claim 1, wherein the fragment of FLIL33 is a discontiguous fragment.

9. The IL-33 decoy peptide of claim 1, wherein the fragment of FLIL33 is 10-50 amino acids.

10. (canceled)

11. The IL-33 decoy peptide of claim 1, wherein the amino acid sequence that enables the peptide to enter a cell is selected from any one of SEQ ID NOS: 15-61.

12. The IL-33 decoy peptide of claim 1, wherein the decoy peptide comprises SEQ ID NO:9 or SEQ ID NO:66.

13. The IL-33 decoy peptide of claim 1, wherein the decoy peptide comprises SEQ ID NO:11 or SEQ ID NO:67.

14. The IL-33 decoy peptide of claim 1, further comprising one or more epitope tags and/or purification tags, wherein the epitope tag is selected from the group consisting of a Myc tag, a FLAG tag, a hemagglutinin (HA) tag and combinations thereof, wherein the purification tag is selected from the group consisting of a histidine tag (6x), a glutathione S-transferase tag and a combination thereof.

15. (canceled)

16. (canceled)

17. The IL-33 decoy peptide of claim 16, further comprising an enzymatic cleavage site.

18. A nucleic acid molecule encoding an IL-33 decoy peptide of claim 1.

19. A viral vector comprising the nucleic acid of claim 18.

20. A pharmaceutical composition comprising the IL-33 decoy peptide of claim 1, and one or more pharmaceutically acceptable excipients.

21. A method of treating and/or preventing a disease or condition that is modulated by IL-33 in a subject in need thereof, comprising administering to the subject an effective amount of the pharmaceutical composition of claim 20.

22. The method of claim 21, wherein the disease or condition is an infection, a viral infection metabolic disorder, or characterized by inflammation or fibrosis.

23. (canceled)

24. (canceled)

25. (canceled)

26. (canceled)

27. The method of claim 21, wherein the decoy peptide disrupts an association of FLIL33 with IPO5.
